(19) United States
(12) Patent Application Publication
(10) Pub. No.: US 2017/0368055 A1
(43) Pub. Date: Dec. 28, 2017

(54) METHODS FOR TREATING INFECTIONS

(71) Applicant: Melinta Therapeutics, Inc., New Haven, CT (US)

(72) Inventors: Danping LI, Middlebury, CT (US); Scott J. HOPKINS, Medina, TX (US); Jarrod LONGCOR, East Haven, CT (US)

(21) Appl. No.: 15/696,603

(22) Filed: Sep. 6, 2017

Related U.S. Application Data
(63) Continuation of application No. 14/744,671, filed on Jun. 19, 2015, now abandoned.
(60) Provisional application No. 62/034,468, filed on Aug. 7, 2014.
(60) Provisional application No. 62/014,790, filed on Jun. 20, 2014.

(51) Int. Cl. A61K 31/4709; A61K 9/00; A61K 9/46; A61K 31/133; A61K 9/20

(52) U.S. Cl. CPC A61K 31/4709; A61K 31/133; A61K 9/2086; A61K 9/0007; A61K 9/2009; A61K 9/0019; A61K 9/2054

(57) ABSTRACT

The present invention relates to methods for treating, preventing, or reducing the risk of microbial infections while minimizing adverse gastrointestinal effects using a two-stage dosing regimen comprising about 1 to about 7 days of intravenous administration followed by about 1 to about 14 days of oral administration of an antimicrobial agent.

### REFERENCE TO RELATED APPLICATIONS

This application is a continuation of and claims priority to U.S. patent application Ser. No. 14/744,671, filed on Jun. 19, 2015, which claims the benefit of priority of U.S. Provisional Patent Application No. 62/034,468, filed Aug. 7, 2014, and U.S. Provisional Patent Application No. 62/014,790, filed Jun. 20, 2014, the entire disclosures of which are incorporated herein by reference.

### FIELD OF THE INVENTION

The present invention relates to methods for treating, preventing, or reducing the risk of microbial infections while minimizing adverse gastrointestinal effects.

### BACKGROUND

Since the discovery of penicillin in the 1920s and streptomycin in the 1940s, many new compounds have been discovered or specifically designed for use as antibiotic agents. It was once believed that infectious diseases could be completely controlled or eradicated with the use of such therapeutic agents. However, such beliefs have been shaken by the fact that strains of cells or microorganisms resistant to currently effective therapeutic agents continue to evolve. In fact, virtually every antibiotic agent developed for clinical use has ultimately encountered problems with the emergence of resistant bacteria. For example, resistant strains of Gram-positive bacteria such as methicillin-resistant staphylococci, penicillin-resistant streptococci, and vancomycin-resistant enterococci have developed, which can cause serious and even fatal results for patients infected with such resistant bacteria. Bacteria that are resistant to macrolide antibiotics, i.e. antibiotics based on a 14- to 16-membered lactone ring, have developed. Also, resistant strains of Gram-negative bacteria such as H. influenzae and M. catarrhalis have been identified. See, e.g., F. D. Lowry, “Antimicrobial Resistance: The Example of Staphylococcus aureus,” J. Clin. Invest., 2003, 111 (9), 1265-1273; Gold, H. S. and Moellering, R. C., Jr., “Antimicrobial-Drug Resistance,” N. Engl. J. Med., 1996, 335, 1445-53; “Antibiotics Crisis Prompts Rethinking On Risks, Rewards,” Medscape Mar. 18, 2013.

Even though many new antimicrobial agents have been developed with a wide range of activity against a broad spectrum of microorganisms, the delivery of these agents can present special challenges. To provide therapeutic efficacy, it is generally desired that the antimicrobial agent be administered to the patient to achieve systemic concentrations in the bloodstream or target organs above a minimum inhibitory concentration for a sufficient time against the particular microbial organism or organisms being targeted. Consequently, an antimicrobial agent that otherwise exhibits an effective antimicrobial profile in vitro can be ineffective, or even harmful, unless properly formulated and delivered for in vivo administration.

For example, antimicrobial agents can affect the flora of the gastrointestinal tract, particularly in the small and large intestine, which can result in untoward gastrointestinal side effects such as diarrhea, flatulence, dyspepsia, belching, bloating, gastritis, and general abdominal discomfort. Furthermore, antimicrobial agents can exert a selective pressure on the resident bacteria of the gastrointestinal tract resulting in the emergence of bacteria resistant to the antimicrobial agent. The gastrointestinal side effects can effect patient compliance with the antimicrobial dosing regimen, therefore compromising drug effectiveness and potentially subjecting the patient to recurring or more resistant infections. In severe cases, the gastrointestinal side effects can result in disruption or even total discontinuation of therapy.

The bacterial resistance issuance is a broader public health concern. In certain instances, because of the selective pressure of the antimicrobial agent on the bacterial flora of the gastrointestinal tract, the patient receiving antimicrobial therapy can become colonized by bacteria resistant to the antimicrobial agent. The resultant resistant bacteria can not only be harmful to the patient but can be further disseminated into and become established in the community.

It is believed by those skilled in the art that the gastrointestinal side effects and resistance problems are caused by residual antimicrobial agent that is not absorbed from the gastrointestinal tract into the bloodstream. In such instances, it is believed that the antimicrobial agent that remains in and passes through the gastrointestinal tract, particularly the antimicrobial agent that passes into the cecum and colon, can cause these gastrointestinal side effects and resistance problems. Additionally, the antimicrobial agent that is absorbed into the bloodstream can, depending on the particular pharmacokinetics of the antimicrobial agent, be excreted from the liver through the bile and back into the gastrointestinal tract to further augment the gastrointestinal side effects and resistance problems.

There is thus a need to develop formulations and dosing regiments which minimize the presence of residual or unwanted antimicrobial agent in the gastrointestinal tract. In particular there is a need to develop formulations and dosing regimens which minimize the presence of residual or unwanted antimicrobial agent in the cecum and colon. There is also a need to develop formulations and dosing regimens which minimize the undesirable gastrointestinal side effects and the development of antibiotic resistance.

The present invention relates to methods and pharmaceutical compositions for delivering a quinolone carboxylic acid derivative antimicrobial compound while minimizing adverse gastrointestinal effects. These methods and pharmaceutical compositions have improved gastrointestinal tolerability.

Therefore, the present invention addresses the foregoing and other needs by providing compositions and methods.

### BRIEF DESCRIPTION OF THE FIGURES

FIG. 1 shows a powder X-ray diffraction (XRPD) pattern of crystalline D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboxylate.

FIG. 2 shows a powder X-ray diffraction pattern of crystalline D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboxylate trihydrate.

FIG. 3 shows a Modulated Differential Scanning calorimetry (mDSC) thermogram of crystalline D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate.

### SUMMARY OF THE INVENTION

The present invention is based, in part, on the surprising discovery that a dosing regimen of a quinolone carboxylic acid derivative antimicrobial agent in which intravenous dosing of an antimicrobial agent followed by oral dosing of the antimicrobial agent provides improved gastrointestinal tolerability of the antimicrobial agent. These compositions are useful for oral administration, for treating, preventing, or reducing the risk of microbial infections.

The present invention relates to methods for treating, preventing, or reducing the risk of microbial infections while minimizing adverse gastrointestinal effects. In one aspect, the method comprises administering a quinolone carboxylic acid derivative antimicrobial agent according to a regimen comprising about 1 to about 7 days of intravenous administration followed by about 1 to about 14 days of oral administration on a once daily or twice daily schedule.

It is understood that any of the embodiments described below can be combined in any desired way, and any embodiment or combination of embodiments can be applied to each of the aspects described below, unless clearly exclusive.

In one aspect, the invention provides a method for treating, preventing, or reducing the risk of a microbial infection in a patient in need thereof comprising: (a) intravenously administering a pharmaceutically effective amount of a quinolone carboxylic acid derivative or a pharmaceutically acceptable salt or ester thereof according to a continuous schedule from about 1 to about 7 days; and thereafter (b) orally administering a pharmaceutically effective amount of said quinolone carboxylic acid derivative or said pharmaceutically acceptable salt or ester thereof according to a continuous schedule of once daily or twice daily from about 1 to about 14 days.

In one aspect, the invention provides a method for treating, preventing, or reducing the risk of a microbial infection in a patient in need thereof comprising: (a) intravenously administering a pharmaceutically effective amount of a quinolone carboxylic acid derivative or a pharmaceutically acceptable salt or ester thereof according to a continuous schedule from about 1 to about 4 days; and thereafter (b) orally administering a pharmaceutically effective amount of said quinolone carboxylic acid derivative or said pharmaceutically acceptable salt or ester thereof according to a continuous schedule of once daily or twice daily from about 1 to about 7 days. In some embodiments, the invention provides a method for treating a bacterial infection in a patient in need thereof as described herein. In some embodiments, the invention provides a method for preventing a bacterial infection in a patient in need thereof as described herein. In some embodiments, the invention provides a method for reducing the risk of a bacterial infection in a patient in need thereof as described herein.

In some embodiments, the intravenous administration is from about 1 to about 6 days. In some embodiments, the intravenous administration is from about 1 to about 5 days. In some embodiments, the intravenous administration is from about 1 to about 4 days. In some embodiments, the intravenous administration is from about 1 to about 3 days. In some embodiments, the intravenous administration is from about 1 to about 2 days.

In some embodiments, the oral administration is from about 1 to about 13 days. In some embodiments, the oral administration is from about 1 to about 12 days. In some embodiments, the oral administration is from about 1 to about 11 days. In some embodiments, the oral administration is from about 1 to about 10 days. In some embodiments, the oral administration is from about 1 to about 9 days. In some embodiments, the oral administration is from about 1 to about 8 days. In some embodiments, the oral administration is from about 1 to about 7 days. In some embodiments, the oral administration is from about 1 to about 6 days. In some embodiments, the oral administration is from about 1 to about 5 days. In some embodiments, the oral administration is from about 1 to about 4 days. In some embodiments, the oral administration is from about 1 to about 3 days.

In some embodiments, the method provides a reduced potential for adverse gastrointestinal side effects relative to oral administration.

In some embodiments, the method provides improved gastrointestinal tolerability relative to oral administration.

In some embodiments, the method provides a reduced potential to cause bacterial resistance in the gastrointestinal tract relative to oral administration.

In some embodiments, the quinolone carboxylic acid derivative corresponds to the following structure of Formula 1

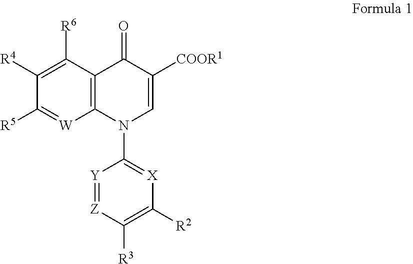

wherein R¹ represents a hydrogen atom or a carboxyl protective group;
R² represents a hydroxyl group, a lower alkoxy group, or a substituted or unsubstituted amino group;
R³ represents a hydrogen atom or a halogen atom;
R⁴ represents a hydrogen atom or a halogen atom;
R⁵ represents a halogen atom or an optionally substituted saturated cyclic amino group;
R⁶ represents a hydrogen atom, a halogen atom, a nitro group, or an optionally protected amino group;
X, Y and Z may be the same or different and respectively represent a nitrogen atom, —CH═ or —CR⁷═;
R⁷ represents a lower alkyl group, a halogen atom, or a cyano group,
with the proviso that at least one of X, Y and Z represent a nitrogen atom, and W represents a nitrogen atom or —CR⁸═;
wherein R⁸ represents a hydrogen atom, a halogen atom, or a lower alkyl group.

In some embodiments, when R¹ represents a hydrogen atom, R² represents an amino group, R³ and R⁴ represent a fluorine atom, R⁶ represents a hydrogen atom, X represents a nitrogen atom, Y represents —CR⁷═ wherein R⁷ represents a fluorine atom, Z represents —CH═, and W is —CR⁸═ wherein R⁸ represents a chlorine atom, then R⁵ is not a 3-hydroxyazetidine-1-yl group.

In some embodiments, the quinolone carboxylic acid derivative corresponds to the following compound (A)

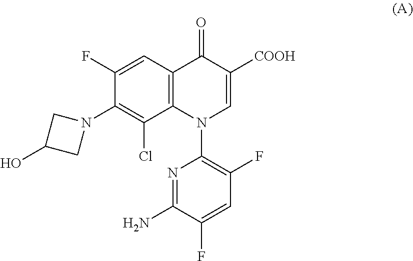 [SMILES: Nc1nc(-n2cc(C(=O)O)c(=O)c3cc(F)c(N4CC(O)C4)c(Cl)c32)c(F)cc1F]

or a pharmaceutically acceptable salt or ester thereof.

In some embodiments, the pharmaceutically acceptable salt of said quinolone carboxylic acid derivative is a D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate.

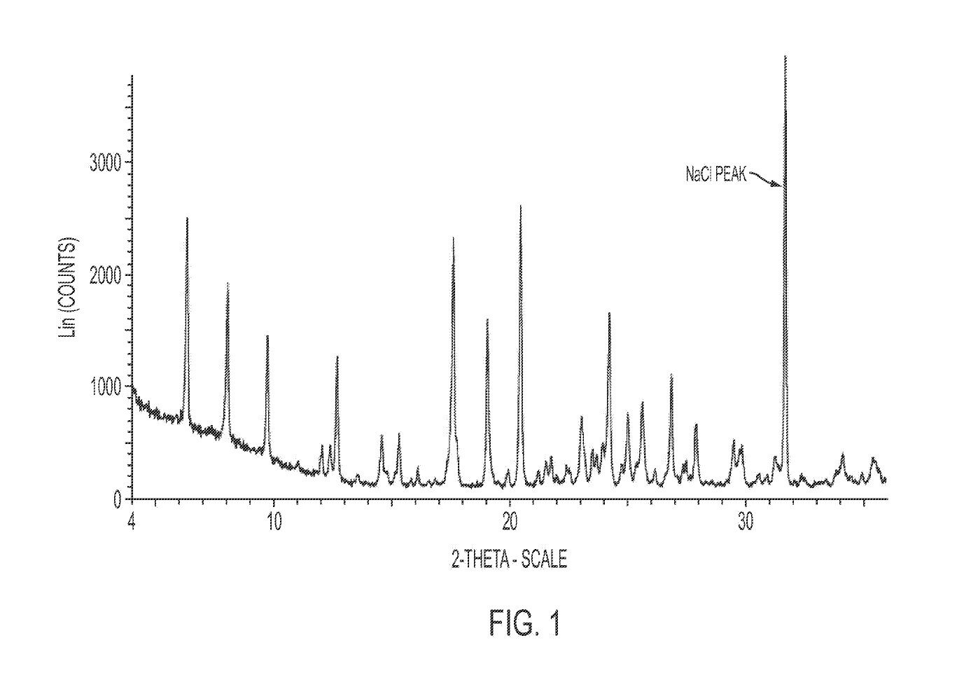

In some embodiments, the pharmaceutically acceptable salt of said quinolone carboxylic acid derivative is a crystalline D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate characterized, wherein the pattern is obtained from a copper radiation source with Cu—Kα radiation, by the x-ray powder diffraction pattern shown in FIG. 1.

In some embodiments, the pharmaceutically acceptable salt of said quinolone carboxylic acid derivative is D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate.

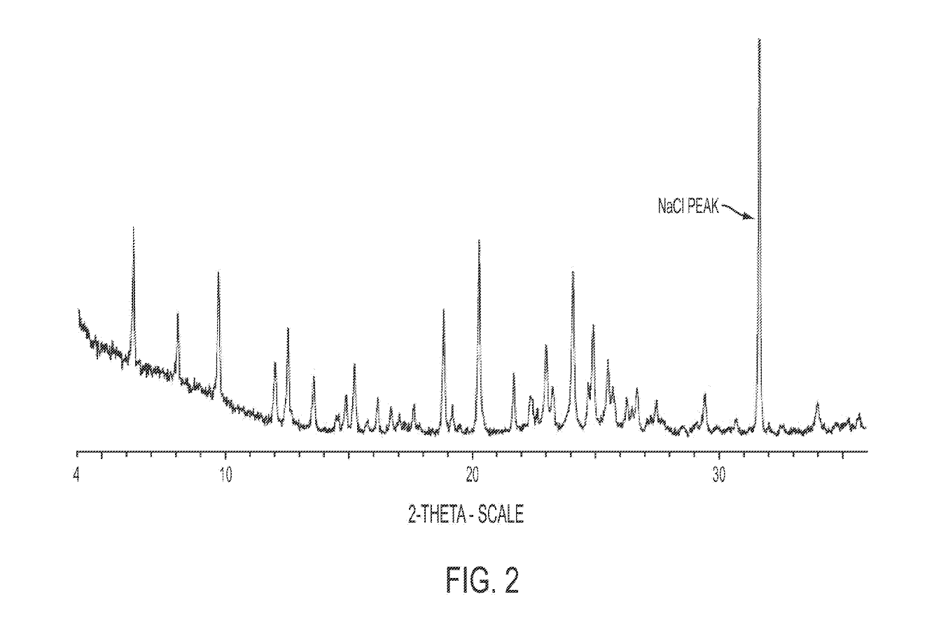

In some embodiments, the pharmaceutically acceptable salt of said quinolone carboxylic acid derivative is crystalline D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate, characterized, when measured at about 25° C. with Cu—Kα radiation, by the x-ray powder diffraction pattern shown in FIG. 2.

In some embodiments, the formulation intravenously administered comprises from about 0.1 mg to about 1500 mg of delafloxacin, on an acid active basis. In some embodiments, the formulation intravenously administered comprises from about 100 mg to about 750 mg of delafloxacin, on an acid active basis. In some embodiments, the formulation intravenously administered comprises from about 250 to about 500 mg of delafloxacin, on an acid active basis. In some embodiments, the formulation intravenously administered comprises about 300 mg of delafloxacin, on an acid active basis. In some embodiments, the formulation intravenously administered comprises about 400 mg of delafloxacin, on an acid active basis. In some embodiments, the formulation intravenously administered comprises about 450 mg of delafloxacin, on an acid active basis. In some embodiments, the formulation intravenously administered comprises from about 0.1 to about 10 mg, from about 10 mg to about 20 mg, from about 20 mg to about 50 mg, from about 50 mg to about 100 mg, from about 100 mg to about 200 mg, from about 200 mg to about 500 mg, from about 500 mg to about 1000 mg of delafloxacin or from about 1000 mg to about 1500 mg of delafloxacin, on an acid active basis. In some embodiments, the formulation intravenously administered comprises about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg of delafloxacin, on an acid active basis.

In some embodiments, the formulation orally administered comprises from about 0.1 mg to about 1500 mg of delafloxacin, on an acid active basis. In some embodiments, the formulation orally administered comprises from about 100 mg to about 750 mg of delafloxacin, on an acid active basis. In some embodiments, the formulation orally administered comprises from about 250 to about 500 mg of delafloxacin, on an acid active basis. In some embodiments, the formulation orally administered comprises about 300 mg of delafloxacin, on an acid active basis. In some embodiments, the formulation orally administered comprises about 400 mg of delafloxacin, on an acid active basis. In some embodiments, the formulation orally administered comprises about 450 mg of delafloxacin, on an acid active basis. In some embodiments, the formulation orally administered comprises from about 0.1 to about 10 mg, from about 10 mg to about 20 mg, from about 20 mg to about 50 mg, from about 50 mg to about 100 mg, from about 100 mg to about 200 mg, from about 200 mg to about 500 mg, from about 500 mg to about 1000 mg of delafloxacin or from about 1000 mg to about 1500 mg of delafloxacin, on an acid active basis. In some embodiments, the formulation orally administered comprises about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg of delafloxacin, on an acid active basis.

In some embodiments, the formulation orally administered is in the form of a tablet. In some embodiments, the tablet is a single layer tablet. In further embodiments, the tablet is a bilayer tablet.

In some embodiments, the formulation orally administered is in the form of a capsule.

### DETAILED DESCRIPTION OF THE INVENTION

The present invention relates to methods for treating, preventing, or reducing the risk of microbial infections while minimizing adverse gastrointestinal effects. These methods can comprise administering a quinolone carboxylic acid derivative antimicrobial agent according to a regimen comprising about 1 to about 7 days of intravenous administration followed by about 1 to about 14 days of oral administration on a once daily or twice daily schedule.

In the present invention, it has surprisingly been discovered that the combination of a dosing regimen comprising about 1 to about 7 days of intravenous administration of a quinolone carboxylic acid derivative followed by about 1 to about 14 days of oral administration of the quinolone carboxylic acid derivative on a once daily or twice daily schedule is effective for treating, preventing, or reducing the risk of microbial infections while minimizing adverse gastrointestinal effects.

The present invention relates to a two-stage, or intravenous oral switch regimen, comprising an intravenous administration phase followed by an oral administration phase.

In other embodiments, the present invention relates to a method for treating, preventing, or reducing the risk of a bacterial infection comprising administering to a patient in need thereof a composition as described herein. In other embodiments, the present invention relates to a method for treating, preventing, or reducing the risk of a bacterial infection in a patient in need thereof, while reducing the potential for adverse gastrointestinal side effects. In other embodiments, the present invention relates to a method for treating, preventing, or reducing the risk of a bacterial infection in a patient in need thereof, while reducing the potential for developing resistant microbial organisms.

1. Definitions

The term “patient”, as used herein, means the human or animal (in the case of an animal, more typically a mammal) subject. The term “mammal” includes a human, mouse, rat, guinea pig, dog, cat, horse, cow, pig, or non-human primate, such as a monkey, chimpanzee, baboon or rhesus. In one embodiment, the mammal is a human. The patient is usually one that is in need of the compositions or methods described herein. “In need of,” can mean that the patient has or is diagnosed as having an infection, e.g., a microbial infection, or that the patient is at risk of contracting an infection due to an injury, a medical or surgical procedure, or microbial exposure, or could be in a position that could subject the patient to such exposure. Such infections can be due to, e.g., a skin infection, nosocomial pneumonia, post-viral pneumonia, an abdominal infection, a urinary tract infection, bacteremia, septicemia, endocarditis, an atrio-ventricular shunt infection, a vascular access infection, meningitis, infection due to surgical or invasive medical procedures, a peritoneal infection, a bone infection, a joint infection, a methicillin-resistant Staphylococcus aureus infection, a vancomycin-resistant Enterococci infection, a linezolid-resistant organism infection, tuberculosis, a quinolone resistant Gram-positive infection, a ciprofloxacin resistant methicillin resistant (MRSA) infection, bronchitis, a complicated skin and skin structure infection (cSSSI), an uncomplicated skin and skin structure infection (uSSSI), a community respiratory-tract infection, and a multi-drug resistant (MDR) Gram-negative infection.

The term “preventing”, as used herein, means, e.g., to completely or almost completely stop an infection from occurring, for example when the patient is predisposed to an infection or at risk of contracting an infection.

The term “reducing the risk of”, as used herein means, e.g., to lower the likelihood or probability of an infection occurring, for example when the patient is predisposed to an infection or at risk of contracting an infection.

The term “treating” as used herein means, e.g., to cure, inhibit, arrest the development, relieve the symptoms or effects of, ameliorating, or cause the regression of an infection in a patient having an infection.

It should be recognized that the terms “preventing”, “reducing the risk of”, and “treating” are not intended to limit the scope of the invention and that there can be overlap amongst these terms.

As used herein, the term “effective amount” means an amount of a pharmaceutically active compound, i.e. a drug active, e.g., a quinolone carboxylic acid derivative or pharmaceutically acceptable salt thereof, given to a recipient patient sufficient to elicit biological activity, for example, anti-infective activity, e.g., anti-microbial activity.

The term “prophylactically effective amount” means an amount of a pharmaceutically active compound, i.e. a drug active, e.g., a quinolone carboxylic acid derivative given to a recipient patent sufficient to prevent or reduce the risk of a microbial infection.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In one embodiment, non-aqueous media, for example ether, ethyl acetate, ethanol, isopropanol, or acetonitrile are useful for forming salts of the present compounds. Lists of suitable salts are found in Remington's Pharmaceutical Sciences, 18^{th} ed. (Mack Publishing Company, 1990). For example, salts can include, but are not limited to, the hydrochloride and acetate salts of the aliphatic amine-containing, hydroxyl amine-containing, and imine-containing compounds of the present invention.

Additionally, the compounds of the present invention, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

As used herein, “pharmaceutically acceptable esters” refer to derivatives of the disclosed compounds wherein the parent compound is modified by an alcohol ester of a carboxylic acid or a carboxylic acid ester of an alcohol. The compounds of the present invention can also be prepared as esters, for example pharmaceutically acceptable esters. For example a carboxylic acid function group in a compound can be converted to its corresponding ester, e.g., a methyl, ethyl, or other ester. Also, an alcohol group in a compound can be converted to its corresponding ester, e.g., an acetate, propionate, or other ester. In the present invention, particularly useful esters are the C₁-C₆ alkyl alcohol esters, i.e. the C₁-C₆ straight, branched, and cyclic alkyl esters, the phenyl ester, and the benzyl ester. Examples of these esters include the methyl ester, the ethyl ester, the n-propyl ester, the isopropyl ester, the n-butyl ester, and so forth.

As used herein, the term “unit dosage” or “unit dosage form”, means a single dose of a pharmaceutical composition that is intended to be administered in its entirety. A unit dosage or a unit dosage form is a convenient form for administering a premeasured amount of a drug active.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

2. Compositions of the Present Invention

The compositions of the present invention comprise all or some of the following components. The compositions can be defined either prior to or after mixing of the components.

Suitable components are described in e.g., Eds. R. C. Rowe, et al., Handbook of Pharmaceutical Excipients, Fifth Edition, Pharmaceutical Press (2006); Remington's Pharmaceutical Sciences, 18^{th} ed. (Mack Publishing Company, 1990); and Remington: The Science and Practice of Pharmacy, 20^{th} Edition, Baltimore, Md.: Lippincott Williams & Wilkins, 2000, which are incorporated by reference herein in their entirety. Even though a functional category can be provided for many of these carrier components, such a functional category is not intended to limit the function or scope of the component, as one of ordinary skill in the art will recognize that a component can belong to more than one functional category and that the level of a specific component and the presence of other components can affect the functional properties of a component.

a. Compositions for Intravenous (I.V.) Administration

PCT Application publication number WO 2010/056782 A2, to Rib-X Pharmaceuticals, Inc., entitled Antimicrobial Compositions, published May 20, 2010, which is incorporated by reference herein in its entirety, describes the intravenous formulation composition.

i. Quinolone Carboxylic Acid Derivative

The compositions of the present invent comprise a quinolone carboxylic acid derivative, (alternatively known as, inter alia, a pyridonecarboxylic acid derivative or a pyridone carboxylic acid derivative), or a pharmaceutically acceptable salt thereof, as an antimicrobial compound, i.e. as the active pharmaceutical ingredient, or API, of the compositions of the present invention. The invention further provides methods for synthesizing any one of the compounds of the present invention. The invention also provides pharmaceutical compositions comprising an effective amount of one or more of the compounds of the present invention and a pharmaceutically acceptable carrier. The present invention further provides methods for making these compounds, carriers, and pharmaceutical compositions.

Quinolone carboxylic acid derivatives, useful herein are described, including their syntheses, formulation, and use, in U.S. Pat. No. 6,156,903, to Yazaki et al., issued Dec.5, 2000 and its certificates of correction of Nov. 13, 2001 and Dec. 11, 2001; U.S. Pat. No. 6,133, 284, to Yazaki et al., issued Oct. 17, 2000; U.S. Pat. No. 5,998, 436, to Yazaki et al., issued Dec. 7, 1999 and its certificates of correction of Jan. 23, 2001, Oct. 30, 2001, and Dec. 17, 2002; PCT Application No. WO 2006/110815, to Abbott Laboratories, published Oct. 19, 2006; PCT Application No. WO 2006/042034, to Abbott Laboratories, published Apr. 20, 2006, PCT Application No. WO 2006/015194, to Abbott Laboratories, published Feb. 9, 2006; PCT Application No. WO 01/34595, to Wakunaga Pharmaceutical Co., Ltd., published May 17, 2001; and PCT Application No. WO 97/11068, to Wakunaga Pharmaceutical Co., Ltd., published Mar. 27, 1997, the contents of each of which are hereby incorporated by reference in their entireties.

Quinolone carboxylic acid derivatives useful in the methods, compositions, and uses of the present invention include compounds corresponding to Formula 1

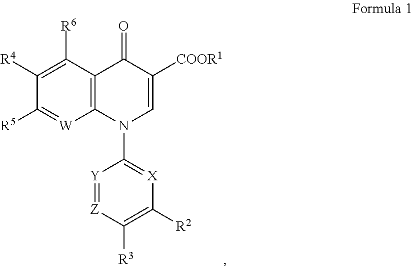

wherein with respect to Formula 1, R¹ represents a hydrogen atom or a carboxyl protective group; R² represents a hydroxyl group, a lower alkoxy group, or a substituted or unsubstituted amino group; R³ represents a hydrogen atom or a halogen atom; R⁴ represents a hydrogen atom or a halogen atom; R⁵ represents a halogen atom or an optionally substituted saturated cyclic amino group; R⁶ represents a hydrogen atom, a halogen atom, a nitro group, or an optionally protected amino group; X, Y and Z may be the same or different and respectively represent a nitrogen atom, —CH═ or —CR⁷═ (wherein R⁷ represents a lower alkyl group, a halogen atom, or a cyano group), with the proviso that at least one of X, Y and Z represent a nitrogen atom, and W represents a nitrogen atom or —CR⁸═ (wherein R⁸ represents a hydrogen atom, a halogen atom, or a lower alkyl group).

In one embodiment, when R¹ represents a hydrogen atom, R² represents an amino group, R³ and R⁴ represent a fluorine atom, R⁶ represents a hydrogen atom, X represents a nitrogen atom, Y represents —CR⁷═ (wherein R⁷ represents a fluorine atom), Z represents —CH═, and W is —CR⁸═ (wherein R⁸ represents a chlorine atom), then R⁵ is not a 3-hydroxyazetidine-1-yl group; or a pharmaceutically acceptable salt, ester, or prodrug thereof.

When R¹ is a carboxyl protective group, it may be any carboxylate ester residue which cleaves relatively easily, such as in vivo, to generate the corresponding free carboxyl group. Exemplary carboxyl protective groups include those which may be eliminated by hydrolysis, catalytic reduction, and other treatments under mild conditions such as lower alkyl groups such as methyl group, ethyl group, n-propyl group, i-propyl group, n-butyl group, i-butyl group, t-butyl group, pentyl group, hexyl group, and heptyl group; lower alkenyl groups such as vinyl group, allyl group, 1-propenyl group, butenyl group, pentenyl group, hexenyl group, and heptenyl group; aralkyl groups such as benzyl group; and aryl groups such as phenyl group and naphthyl group; and those which may be readily eliminated in the body such as lower alkanoyloxy lower alkyl groups such as acetoxymethyl group and pivaloyloxymethyl group; lower alkoxycarbonyloxy lower alkyl group such as methoxycarbonyloxymethyl group and 1-ethoxycarbonyloxyethyl group; lower alkoxymethyl group such as methoxymethyl group; lactonyl group such as phthalidyl; di-lower alkylamino lower alkyl group such as 1-dimethylaminoethyl group; and (5-methyl-2-oxo-1,3-dioxole-4-yl)methyl group.

In one embodiment, R¹ in Formula 1 is H.

In one embodiment, R² in Formula 1 is —NH₂.

In one embodiment, R³ in Formula 1 is halogen.

In another embodiment, R³ in Formula 1 is fluorine.

In one embodiment, R⁴ in Formula 1 is halogen.

In another embodiment, R⁴ in Formula 1 is fluorine.

In one embodiment, R⁵ in Formula 1 is a substituted cyclic amino group.

In one embodiment, R⁵ in Formula 1 is

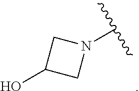 [SMILES: *N1CC(O)C1]

In one embodiment, R⁶ in Formula 1 is hydrogen.

In one embodiment, X in Formula 1 is a nitrogen atom.

In one embodiment, Y in Formula 1 is ═CR⁷—.

In one embodiment, R⁷ in Formula 1 is a halogen.

In another embodiment, R⁷ in Formula 1 is fluorine.

In one embodiment, Z in Formula 1 is ═CH—.

In one embodiment, W in Formula 1 is ═CR⁸—.

In one embodiment, R⁸ in Formula 1 is a halogen.

In another embodiment, R⁸ in Formula 1 is chlorine.

It is noted that the substituents R¹, R², R³, R⁴, R⁵, R⁶, R⁷, R⁸, R⁹, A, J¹, J², J³, W, X, Y, Z, e, f, and g are defined herein for convenience with respect to the chemical structure for the quinolone carboxylic acid derivatives, for example for Formula 1.

In other embodiments, the present invention relates to a method, composition, or use for a compound of Formula 1, wherein W is —CR⁸═, wherein R⁸ represents a hydrogen atom, a halogen atom, or a lower alkyl group.

In other embodiments, the present invention relates to a method, composition, or use for a quinolone carboxylic acid derivative of Formula 1, wherein R⁵ is a group represented by the following formula (a) or (b):

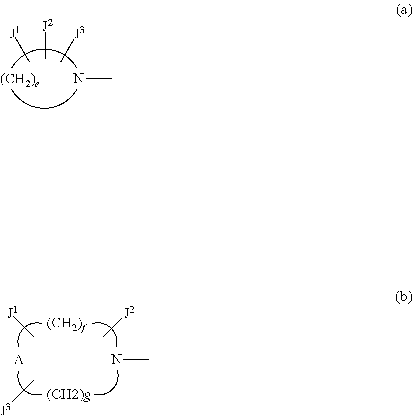

wherein A represents an oxygen atom, sulfur atom or NR⁹ (wherein R⁹ represents hydrogen atom or a lower alkyl group), e represents a number from 3 to 5, f represents a number from 1 to 3, g represents a number from 0 to 2, J¹, J² and J³, which may be the same or different from one another, represent a hydrogen atom, hydroxyl group, lower alkyl group, amino lower alkyl group, amino group, lower alkylamino group, lower alkoxy group, or a halogen atom.

In other embodiments, the present invention relates to a method, composition, or use for a quinolone carboxylic acid derivative of Formula 1, wherein R⁵ is a group represented by formula (a):

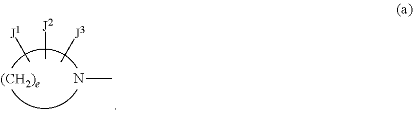

In other embodiments, the present invention relates to a method, composition, or use for a quinolone carboxylic acid derivative of structure Quinolone Carboxylic Acid Derivative of Formula 1, wherein e in the formula (a) is 3 or 4:

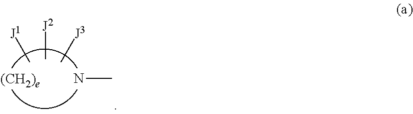

In other embodiments, the present invention relates to a method, composition, or use for a quinolone carboxylic acid derivative of structure Quinolone Carboxylic Acid Derivative of Formula 1, wherein R¹ is a hydrogen atom; R² is an amino group, lower alkylamino group, or a di-lower alkylamino group; R³ is a halogen atom; R⁴ is a halogen atom; R⁶ is hydrogen atom; X is a nitrogen atom; Y and Z are —CH═ or —CR⁷═ (wherein R⁷ is a lower alkyl group or a halogen atom); and W is —CR⁸═ (wherein R⁸ is a halogen atom or a lower alkyl group).

In other embodiments, the present invention relates to a method, composition, or use for a quinolone carboxylic acid derivative of structure Quinolone Carboxylic Acid Derivative of Formula 1, wherein R² is amino group; R³ is fluorine atom; R⁴ is a fluorine atom; Y is —CF═; Z is —CH═; W is —CR⁸═ (wherein R⁸ is a chlorine atom, bromine atom or a methyl group), and e in formula (a) is 3.

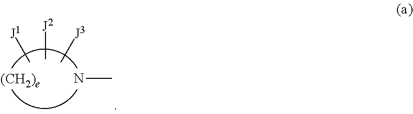

In other embodiments, the present invention relates to a method, composition, or use wherein said quinolone carboxylic acid corresponds to the compound (A):

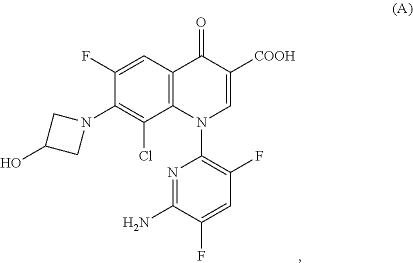 [SMILES: Nc1nc(-n2cc(C(=O)O)c(=O)c3cc(F)c(N4CC(O)C4)c(Cl)c32)c(F)cc1F]

or a pharmaceutically acceptable salt, ester, or prodrug thereof. This foregoing quinolone carboxylic acid derivative, compound (A), is also known by the USAN, delafloxacin, the publicly disclosed code names RX-3341, ABT-492 and WQ 3034, and also by, inter alia, the chemical name 1-(6-amino-3,5-difluoro-2-pyridinyl)-8-chloro-6-fluoro-1,4-dihydro-7-(3-hydroxy-1-azetidinyl)-4-oxo-3-quinolinecarboxylic acid or 1-(6-amino-3,5-difluoro-2-pyridinyl)-8-chloro-6-fluoro-1,4-dihydro-7-(3-hydroxyazetidin-1-yl)-4-oxo-3-quinolinecarboxylic acid. This carboxylic acid form of the compound corresponds to the CAS Registry Number 189279-58-1. Furthermore, WO 2006/042034, cited above discloses the 1-deoxy-1-(methylamino)-D-glucitol salt of this compound, also known as D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate, and the trihydrate of the 1-deoxy-1-(methylamino)-D-glucitol salt of this compound, also known as D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate. The 1-deoxy-1-(methylamino)-D-glucitol salt and the 1-deoxy-1-(methylamino)-D-glucitol salt trihydrate correspond to the CAS Registry Numbers 352458-37-8 and 883105-02-0, respectively. 1-Deoxy-1-(methylamino)-D-glucitol corresponds to the CAS Registry Number 6284-40-8. 1-Deoxy-1-(methylamino)-D-glucitol is also known by the name meglumine. D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate, which is the meglumine salt of delafloxacin, is also known as delafloxacin meglumine. D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate, which is the trihydrate of the meglumine salt of delafloxacin, is also known as delafloxacin meglumine trihydrate. WO 2006/042034 also discloses a crystalline form of the 1-deoxy-1-(methylamino)-D-glucitol salt characterized when measured at about 25° C. with Cu—Kα radiation, by the x-ray powder diffraction pattern disclosed therein and a crystalline form of the 1-deoxy-1-(methylamino)-D-glucitol salt trihydrate when measured at about 25° C. with Cu—Kα radiation, by the x-ray powder diffraction pattern shown in FIG. 2 (see WO 2006/042034, which is hereby incorporated by reference in its entirety). These 1-deoxy-1-(methylamino)-D-glucitol salts are useful in the present invention. Also, see A. R. Haight et al., “Synthesis of the Quinolone ABT-492: Crystallizations for Optimal Processing”, Organic Process Research & Development (2006), 10(4), 751-756, which is hereby incorporated by reference in its entirety.

Additionally other pharmaceutically acceptable salts of the forgoing compound, delafloxacin, include the potassium salt and the tris salt. Tris is a common abbreviation for tris(hydroxymethyl)aminomethane, which is known by the IUPAC name 2-Amino-2-hydroxymethyl-propane-1,3-diol.

In some embodiments, the quinolone carboxylic acid derivative is present in about 0.01% to about 50% by weight of the composition. In further embodiments, the quinolone carboxylic acid derivative comprises from about 0.25% to about 20% by weight of the composition. In yet further embodiments, the quinolone carboxylic acid derivative comprises from about 0.5% to about 10% by weight of the composition. In yet further embodiments, the quinolone carboxylic acid derivative comprises from about 1% to about 5% by weight of the composition. The weight percentage of the quinolone carboxylic acid derivative is determined on an active weight basis of the parent compound. In other words, appropriate adjustments and calculations well known to one of ordinary skill in the art can be readily performed to determine the active weight basis. As a non-limiting example, if the parent free carboxylic acid of delafloxacin, i.e. 1-(6-amino-3,5-difluoro-2-pyridinyl)-8-chloro-6-fluoro-1,4-dihydro-7-(3-hydroxy-1-azetidinyl)-4-oxo-3-quinolinecarboxylic acid, is used, its weight would have to be adjusted if a salt such as the sodium salt were to be used, because the molecular weight of the compound would increase by about 21.9, although the amount of active compound delivered is the same.

In some embodiments, the quinolone carboxylic acid derivative is present in the pharmaceutical compositions described herein in the amount of from about 0.1 mg to about 1500 mg. In some embodiments, the quinolone carboxylic acid derivative is present in the pharmaceutical compositions described herein in the amount of from about 100 mg to about 750 mg. In some embodiments, the quinolone carboxylic acid derivative is present in the pharmaceutical compositions described herein in the amount of from about 250 to about 500 mg. In some embodiments, the quinolone carboxylic acid derivative is present in the pharmaceutical compositions described herein in the amount of about 300 mg. In some embodiments, the quinolone carboxylic acid derivative is present in the pharmaceutical compositions described herein in the amount of about 400 mg. In some embodiments, the quinolone carboxylic acid derivative is present in the pharmaceutical compositions described herein in the amount of about 450 mg. In some embodiments, the quinolone carboxylic acid derivative is present in the pharmaceutical compositions described herein in the amount of from about 0.1 to about 10 mg, from about 10 mg to about 20 mg, from about 20 mg to about 50 mg, from about 50 mg to about 100 mg, from about 100 mg to about 200 mg, from about 200 mg to about 500 mg, from about 500 mg to about 1000 mg or from about 1000 mg to about 1500 mg. In some embodiments, the quinolone carboxylic acid derivative is present in the pharmaceutical compositions described herein in the amount of about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg.

The dose of the pharmaceutical active and mode of administration of the pharmaceutical composition will depend upon the intended patient or subject and the targeted microorganism, e.g., the target bacterial organism.

As further described below, it is often advantageous to mill the pharmaceutical active to a small and uniform particle size, usually in the micron range, i.e. micronization. Milling can be performed using standard techniques well known to one of ordinary skill in the art. In one embodiment, useful particle size ranges for the pharmaceutical active are generally from about 0.01 microns to about 100 microns. In another embodiment, useful particle size ranges for the pharmaceutical active are from about 0.1 microns to about 20 microns. In another embodiment, useful particle size ranges for the pharmaceutical active are from about 0.5 microns to about 5 microns.

ii. Cyclodextrins

The compositions of the present invention comprise a cyclodextrin (sometimes abbreviated as “CD”). Cyclodextrins are cyclic oligosaccharides composed of five or more alpha-D-glucopyranoside units, i.e. sugar units. Cyclodextrins are produced from starch by means of enzymatic conversion. Cyclodextrins having six sugar units are referred to as alpha-cyclodextrins (also “α-cyclodextrins”). Cyclodextrins having seven sugar units are referred to as beta-cyclodextrins (also “β-cyclodextrins”). Cyclodextrins having eight sugar units are referred to as gamma-cyclodextrins (also “γ-cyclodextrins”). Cyclodextrins are further described in the Handbook of Pharmaceutical Excipients, Third Edition, Edited by A. H. Kibbe, pages 165-168, American Pharmaceutical Association and Pharmaceutical Press (2000), which are incorporated by reference herein in their entirety.

Cyclodextrins, which are cyclic oligosaccharides, have been reported for use in pharmaceutical formulations. Also, publications in the field of pharmaceutical product development have reported various formulations and technologies relating to drug solubility and stability, and also to tolerability of intravenous formulations. See, for example, U.S. Pat. No. 6,407,079 B1, to Muller et al., issued Jun. 18, 2002; U.S. Pat. No. 5,874,418, to Stella et al., issued Feb. 23, 1999; U.S. Pat. No. 5,376,645, to Stella et al., issued Dec. 27, 1994; U.S. Pat. No. 5,134,127, to Stella et al., issued Jul. 28, 1992; and U.S. Pat. No. 5,084,276, to Yunker et al., issued Jan. 28, 1992, each of which is hereby incorporated by reference in its entirety.

However, although cyclodextrins have been taught as excipients for formulating pharmaceutical compositions for intravenous administration, not all cyclodextrins are automatically useable to provide the desired formulation characteristics and benefits. Based on what is taught in the literature, one cannot a priori select a cyclodextrin for use with a particular drug product to obtain the desired end result. The final drug product formulation is the result of a complex interplay of solubility, stability, and toleration.

The cyclodextrin comprises from about 0.01% to about 50% by weight of the composition. In further embodiments, the quinolone carboxylic acid derivative comprises from about 0.25% to about 20% by weight of the composition.

In one embodiment, the compositions of the present invention comprise a cyclodextrin selected from the group consisting of an alpha-cyclodextrin, a beta-cyclodextrin, a gamma-cyclodextrin, and mixtures thereof. In one embodiment, the compositions of the present invention comprise a cyclodextrin selected from the group consisting of a beta-cyclodextrin, a gamma-cyclodextrin, and mixtures thereof.

Beta-cyclodextrins useful herein comprise beta-cyclodextrin ethers, beta-cyclodextrin esters, and mixtures thereof. Beta-cyclodextrins are further described in U.S. Pat. No. 6,407,079, to Muller et al., issued Jun. 18, 2002, which is incorporated by reference herein in its entirety. This '079 patent describes these beta-cyclodextrins as corresponding to the following formula (3):

(beta-Cyclodextrin)-OR (3)

in which the residues R are hydrogen or hydroxyalkyl groups and part of the residues R may optionally be alkyl groups, the beta-cyclodextrin ether having a water-solubility of more than 1.8 gin 100 mL water. In one embodiment, the residues R are hydroxyalkyl groups and part of the residues R may optionally be alkyl groups. In another embodiment, the beta-cyclodextrin ether having a water-solubility of more than 1.8 gin 100 mL water. In still another embodiment, the residues R are hydroxyalkyl groups and part of the residues R may optionally be alkyl groups, the beta-cyclodextrin ether having a water-solubility of more than 1.8 g in 100 mL water.

In one embodiment, a partially etherified beta-cyclodextrin of formula 3 is used in which some of the residues R are hydroxyethyl, hydroxypropyl or dihydroxypropyl groups. Optionally part of the residues R may for instance be methyl or ethyl groups. In one embodiment, the use of partially methylated beta-cyclodextrin ethers with 7 to 14 methyl groups in the beta-cyclodextrin molecule, as they are known from German Offenlegungsschrift 31 18 218 do not come under the present invention. In one embodiment, partial ethers of beta-cyclodextrin comprising only alkyl groups (methyl, ethyl) may be suitable in accordance with the invention if they have a low degree of substitution (as defined below) of 0.05 to 0.2.

Beta-cyclodextrin is a compound with ring structure consisting of 7 anhydro glucose units; it is also referred to as cycloheptaamylose. Each of the 7 glucose rings contains in 2-, 3-, and 6-position three hydroxy groups which may be etherified. In the partially etherified beta-cyclodextrin derivatives used according to the invention only part of these hydroxy groups is etherified with hydroxyalkyl groups and optionally further with alkyl groups. When etherifying with hydroxy alkyl groups which can be carried out by reaction with the corresponding alkylene oxides, the degree of substitution is stated as molar substitution (MS), viz. in mole alkylene oxide per anhydroglucose unit, compare U.S. Pat. No. 3,459,731, column 4. In the hydroxyalkyl ethers of beta-cyclodextrin used in accordance with the invention the molar substitution is between 0.05 and 10. In another embodiment, the molar substitution is between 0.2 and 2. In another embodiment, the molar substitution is about 0.25 to about 1.

The etherification with alkyl groups may be stated directly as degree of substitution (DS) per glucose unit which—as stated above—is 3 for complete substitution. Partially etherified beta-cyclodextrins are used within the invention which comprise besides hydroxyalkyl groups also alkyl groups, especially methyl or ethyl groups, up to a degree of substitution of 0.05 to 2.0. In one embodiment, the degree of substitution with alkyl groups is between 0.2 to 1.5. In one embodiment, the degree of substitution with alkyl groups is between about 0.5 and about 1.2.

In one embodiment, he molar ratio of drug to beta-cyclodextrin ether is about 1:6 to 4:1, especially about 1:2 to 1:1. In one embodiment, the complex forming agent is used in a molar excess.

Useful complex forming agents are especially the hydroxyethyl, hydroxypropyl and dihydroxypropyl ether, their corresponding mixed ethers, and further mixed ethers with methyl or ethyl groups, such as methyl-hydroxyethyl, methyl-hydroxypropyl, ethyl-hydroxyethyl and ethyl-hydroxypropyl ether of beta-cyclodextrin.

The preparation of the hydroxyalkyl ethers of beta-cyclodextrin may be carried out using the method of U.S. Pat. No. 3,459,731. Suitable preparation methods for beta-cyclodextrin ethers may further be found in J. Sziejtli et al., Starke 32, 165 (1980) and A. P. Croft and R. A. Bartsch, Tetrahedron 39, 1417 (1983). Mixed ethers of beta-cyclodextrin can be prepared by reacting beta-cyclodextrin in a basic liquid reaction medium comprising an alkali metal hydroxide, water and optionally at least one organic solvent (e.g., dimethoxyethane or isopropanol) with at least two different hydroxyalkylating and optionally alkylating etherifying agents (e.g., ethylene oxide, propylene oxide, methyl or ethyl chloride).

Beta-cyclodextrins useful herein include hydroxypropyl-beta-cyclodextrins.

Examples of hydroxypropyl-beta-cyclodextrins useful herein include Cavitron® W7 HP7 Pharma, CAS Registry Number 128446-35-5, which is a hydroxypropyl-beta-cyclodextrin having seven glucose units and a molecular substitution per anhydro glucose unit of 0.86-1.14 and Cavitron® W7 HP5 Pharma, which is a hydroxypropyl-beta-cyclodextrin having seven glucose units and a molecular substitution per anhydro glucose unit of 0.59-0.73.

A. Sulfoalkyl Ether Cyclodextrin Derivatives

Sulfoalkyl ether cyclodextrins useful herein include sulfoalkyl ether cyclodextrin derivatives further described in U.S. Pat. No. 5,874,418, to Stella et al., issued Feb. 23, 1999; U.S. Pat. No. 5,376,645, to Stella et al., issued Dec. 27, 1994, along with its certificate of correction of May 19, 2008; and U.S. Pat. No. 5,134,127, to Stella et al., issued Jul. 28, 1992, which are incorporated by reference herein in their entirety. These patents describe the sulfoalkyl ether cyclodextrins as follows:

This invention also provides cyclodextrin derivatives suitable for pharmaceutical use. These derivatives are suitable for use as clathrating agents with drugs to provide clathrate complexes which are useful in parenteral and other pharmaceutical formulations. Procedures for making and isolating the cyclodextrin derivatives are also provided.

The sulfoalkyl ether cyclodextrin derivatives of the present invention are functionalized with (C₂₋₆ alkylene)-SO₃⁻ groups, and are thus charged species. The fact that these compounds have been discovered to possess a very low level of toxicity is surprising in light of the prior art's belief that cyclodextrin derivatives must retain electroneutrality to sustain lack of toxicity (cf. Pitha, “Amorphous Water-Soluble” “Third International Symposium on Recent Advances in Drug Delivery Systems, Salt Lake City, Utah, Feb. 23-27, 1987).

The high aqueous solubility of the cyclodextrin derivatives of the present invention, and their resulting lowered nephrotoxicity, is further surprising in light of U.S. Pat. No. 4,727,064's disclosure that to maintain a high level of solubility for cyclodextrin derivatives, a mixture of derivatives should be used.

The aqueous solubility exhibited by the present sulfoalkyl cyclodextrin derivatives appears to be obtained through solvation of the sulfonic acid moieties. Thus heterogeneous mixture of the present cyclodextrin derivatives is not a requirement for the observed enhanced solvation to occur. Although a mixture of sulfoalkyl ether derivatives can be used in accordance with the present invention, such a mixture is not required for enhanced solubility.

In one embodiment, the sulfoalkyl ether cyclodextrin derivatives of this invention have structures represented by formula (1) shown immediately below:

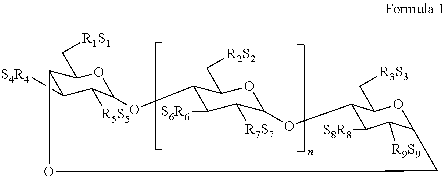

wherein: n is 4, 5 or 6;
R₁, R₂, R₃, R₄, R₅, R₆, R₇, R₈ and R₉ are each independently, O⁻ or a O-(C₂₋₆ alkylene)-SO₃⁻ group, wherein at least one of R₁ and R₂ is independently a O-(C₂₋₆ alkylene)-SO₃⁻ group, for example a O—(CH₂)ₘ—SO₃⁻ group, wherein m is 2 to 6, for example 2 to 4, (e.g., OCH₂CH₂CH₂SO₃⁻ or OCH₂CH₂CH₂CH₂SO₃⁻); and S₁, S₂, S₃, S₄, S₅, S₆, S₇, S₈ and S₉ are each, independently, a pharmaceutically acceptable cation which includes, for example, H⁺, alkali metals (e.g., Li⁺, Na⁺, K⁺), alkaline earth metals (e.g., Ca⁺², Mg⁺²), ammonium ions and amines cations such as the cations C₁₋₆ alkylamines, piperidine, pyrazine, C₁₋₆ alkanolamine and C₄₋₈ cycloalkanolamine.

In another embodiment, R₁ is a O-(C₂₋₆ alkylene)-SO₃⁻ group, for example a O—(CH₂)ₘ—SO₃⁻ group, (e.g., OCH₂CH₂CH₂SO₃⁻ or OCH₂CH₂CH₂CH₂SO₃⁻); R₂ to R₉ are O⁻; and

S₁ to S₉ are as defined for formula 1, supra.

In another embodiment, R₁, R₂ and R₃ are each, independently, a O-(C₁₋₆ alkylene)-SO₃⁻ group, for example a O—(CH₂)ₘSO₃⁻ group, (e.g., OCH₂CH₂CH₂SO₃⁻ or OCH₂CH₂CH₂CH₂SO₃⁻); R₄ to R₉ are O⁻; and S₁ and S₉ are as defined for formula 1, supra.

In another embodiment, R₁ to R₃ are as defined in embodiments (2) or (3); supra; at least one of R₄, R₆ and R₈ is a O-C₂₋₆-alkylene-SO₃⁻ group, for example a O—(CH₂)ₘSO₃⁻ group (e.g., OCH₂CH₂CH₂SO₃⁻ or OCH₂CH₂CH₂CH₂SO₃⁻).

R₅, R₇ and R₉ are O⁻; and S₁ to S₉ are as defined for formula 1, supra.

In another embodiment, R₁, R₂, R₃, R₄, R₆ and R₈ are each, independently, a O-(C₂₋₆-alkylene)-SO₃⁻ group, for example a O—(CH₂)—ₘSO₃⁻ group (e.g., OCH₂CH₂CH₂SO₃⁻ or OCH₂CH₂CH₂CH₂SO₃⁻); R₅, R₇ and R₉ are O⁻; and S₁ to S₉ are as defined for formula 1, supra.

The terms “alkylene” and “alkyl” in this text (e.g., in the O-(C₂₋₆-alkylene)SO₃⁻ group or in the alkylamines) include both linear and branched, saturated and unsaturated (i.e. containing one double bond) divalent alkylene groups and monovalent alkyl groups, respectively. The term “alkanol” in this text likewise includes both linear and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

In one embodiment, the present invention provides compositions containing a mixture of cyclodextrin derivatives having the structure set out in formula (1), where the composition overall contains on the average at least 1 and up to 3n+6 alkylsulfonic acid moieties per cyclodextrin molecule. The present invention also provides compositions containing essentially only one single type of cyclodextrin derivative.

In one embodiment, the present cyclodextrin derivatives are either substituted at least at one of the primary hydroxyl groups (i.e. at least one of R₁ to R₃ is a substituent), or they are substituted at both the primary hydroxyl group and at the 3-position hydroxyl group (i.e. both at least one of R₁ to R₃ and at least one of R₄, R₆ and R₈ are a substituent). In another embodiment, substitution at the 2-position hydroxyl group, while theoretically possible, does not appear to appear to be substantial in the products of the invention. The cyclodextrin derivatives of the present invention are obtained (as discussed below) as purified compositions, for example as compositions containing at least 95 wt. % of cyclodextrin derivative(s) with the substitution occurring at least on the primary hydroxyl group of the cyclodextrin molecule (i.e. R₁, R₂ or R₃ of formula (1)), as determined by 300 MHz ¹H NMR). In an embodiment, purified compositions containing at least 98 wt. % cyclodextrin derivative(s) can be obtained.

In one embodiment, this is to be contrasted with the U.S. Pat. No. 3,426,011 disclosure which reports obtaining only reaction products of the reaction of a cyclodextrin with a sulfone reactant. The reaction products in the '011 patent contain considerable quantities of unsubstituted cyclodextrin starting material.

In one embodiment of compositions of the invention, unreacted cyclodextrin has been substantially removed, with the remaining impurities (i.e. ≦5 wt. % of composition) being inconsequential to the performance of the cyclodextrin derivative-containing composition.

It should be noted that the variables used to describe the cyclodextrins are intended to be separate from the variables used to define the quinolone carboxylic acid derivatives.

The more highly substituted alkyl sulfonic acid cyclodextrin derivatives of the present invention have been discovered to possess, in addition to notably enhanced solubility characteristics and low toxicity, the advantageous property of causing less membrane disruption. In red blood cell hemolysis studies, the more highly substituted cyclodextrin derivatives demonstrated negligible membrane disruption. The mono-substituted cyclodextrin derivatives caused about the same amount of membrane disruption as the hydroxypropyl derivative.

In one embodiment, improved characteristics are achieved by purified compositions of the invention, containing <5%, for example less than 2%, of unreacted beta-cyclodextrin, for example for compositions to be administered to a patient by parenteral administration. In one embodiment, compositions containing somewhat higher amounts of unreacted beta-cyclodextrin, are useful for oral administration.

The allowance for residual beta-cyclodextrin can be broader for a sulfoalkylether cyclodextrin preparation when used in an oral formulation. The oral absorption of beta-cyclodextrin can sometimes be limited (if it occurs at all) and the elimination of beta-cyclodextrin in the feces would preclude any nephrotoxicity. However, the level of beta-cyclodextrin which might be tolerated in an oral formulation would still be dependent upon other characteristics of the material particularly on its intrinsic aqueous solubility.

In one embodiment, the sulfoalkylether cyclodextrins of the present invention may be used for oral formulations, even if unreacted beta-cyclodextrin is contained in an amount of up to about 50%. In one embodiment, the amount is limited to less than 40%. In one embodiment, the amount is limited to less than about 25%.

B. Preparation of the Cyclodextrin (CD) Derivatives

The cyclodextrin derivatives described may be generally prepared by dissolving the cyclodextrin in aqueous base at an appropriate temperature, e.g., 70 degrees to 80 degrees C., at the highest concentration possible. For example, to prepare the cyclodextrin derivatives of an embodiment herein, an amount of an appropriate alkyl sulfone, corresponding to the number of moles of primary CD hydroxyl group present, is added with vigorous stirring to ensure maximal contact of the heterogeneous phase.

To prepare the cyclodextrin derivatives of an embodiment herein, a molar amount of the alkyl sulfone, corresponding to the number of moles of CD used, is used. As would be readily determinable by one of skill in this art, to prepare cyclodextrin derivatives of an embodiment herein, an amount of alkyl sulfone between that stated above is used. Other cyclodextrin derivatives provided by the present invention are prepared Mutatis Mutandis.

The mixtures are allowed to react until one phase results which is indicative of depletion of the alkyl sulfone. The reaction mixture is diluted with an equal volume of water and neutralized with an acid such as hydrochloric acid. The solution is then dialyzed to remove impurities followed by concentration of the solution by ultrafiltration.

The concentrated solution is then subjected to ion-exchange chromatography to remove unreacted cyclodextrin, and then freeze-dried to yield the desired product.

The CD used in this invention may be any CD obtained by known methods, e.g., by the action of cyclodextrin-glucanotransferase (CGTase, E.C., 2.4.1.19.) upon starch. Thus CD herein means alpha-CD in which six glucose units are linked together through alpha-1,4 bond, beta-CD in which seven glucose units are linked together, or gamma-CD in which eight glucose units are linked together, or a mixture thereof. In one embodiment, beta-CD is useful for production of partially derivatized products of broad utility.

As noted herein and depending on the cyclodextrin derivative sought, the amount of alkyl sulfone used as the derivatizing agent should be not more than about one molar equivalent, based on the number of primary hydroxyl groups present in the CD, although the optimum amount may be somewhat dependent on the reactant concentration. Lithium hydroxide, sodium hydroxide and potassium hydroxide may be used as the accelerator. In one embodiment, sodium hydroxide is useful because of its low cost. Its amount must be more than about 30 molar equivalents, and should preferably be in the range of 80 to 200 molar equivalents, with the reactant concentration being set at a level higher than 10% (wt/wt), preferably in the range of 40 to 60% (wt/wt). Any solvent which is substantially inert to the partial alkylation may be used as reaction medium. Typical examples are water, DMF, DMSO, and mixtures thereof. In one embodiment, the use of water alone eases after-treatment. The type and concentration of alkylsulfone and alkali are not critical to the reaction. However, the reaction is normally carried out with stirring at 10° to 80° C. for one hour, or at 20° to 50° C. for 5 to 20 hours.

Techniques commonly used in this field may be employed to isolate and purify the objective compounds from reaction mixtures. These include extraction with organic solvents, dialysis, adsorption chromatography with activated charcoal, silica gel, alumina and other adsorbents, chromatography using, as carrier, cross-linked dextrin, styrene/divinylbenzene copolymers and other cross-linked polymers, and combinations thereof.

Sulfoalkyl ether cyclodextrin derivatives useful herein include sulfobutyl ether cyclodextrins, including sulfobutyl ether beta-cyclodextrins.

An example of a sulfoalkyl ether cyclodextrin derivative useful herein includes Captisol, CAS Registry Number 194615-04-8.

The cyclodextrin comprises from about 1% to about 50% by weight of the composition. In further embodiments, the cyclodextrin comprises from about 5% to about 40% by weight of the composition. In yet further embodiments, the cyclodextrin comprises from about 10% to about 30% by weight of the composition. In yet further embodiments, the cyclodextrin comprises from about 15% to about 25% by weight of the composition.

iii. Water

In one embodiment, the compositions of the present invention comprise from about 0.1% to about 99.9% water, in further embodiments from about 1% to about 99% water, in yet further embodiments from about 5% to about 95% water, and in yet further embodiments from about 10% to about 90% water. In defining a composition, the amount of water can be designated as “q.s.” or “Q.S.”, which means as much as suffices, to provide a final composition or volume of 100%.

iv. Sugars and Sugar Alcohols

The compositions of the present invention, when further made into a lyophile, can further comprise a sugar, a sugar alcohol, or mixtures thereof. Without being limited by theory, these sugars and sugar alcohols are believed to aid in the formation of the lyophile during the lyophilization process. Typically, the lyophile is made by drying the composition under appropriate conditions, such as, for example, by freeze drying. Non-limiting examples of sugars include mannose, sucrose, dextrose, and mixtures thereof. Non-limiting examples of sugar alcohols useful herein include sorbitol, mannitol, xylitol and mixtures thereof.

In one embodiment, the compositions comprise from about 0.1% to about 50% of a sugar or sugar alcohol.

v. Polyhydroxy Amine Compound

In one embodiment, the compositions of the present invention comprise a polyhydroxy amine compound. The polyhydroxy amine compound is separate from and does not encompass the polyhydroxy compound of the compositions of the present invention. The polyhydroxy amine compound is generally a C₃-C₈ straight, branched, or cyclic compound having 2 or more hydroxy substituents, and at least one amine (either substituted or unsubstituted) substituent.

In further embodiments the polyhydroxy amine compound is meglumine. Meglumine corresponds to CAS Registry Number 6284-40-8 and is also known as meglumine, USP; 1-Deoxy-1-(methylamino)-D-glucitol; N-Methyl-D-glucamine; Glucitol, 1-deoxy-1-(methylamino)-, D-(8Cl); Sorbitol, 1-deoxy-1-methylamino-(6Cl); 1-Deoxy-1-(methylamino)-D-glucitol; 1-Deoxy-1-methylaminosorbitol; D-(−)-N-Methylglucamine; Meglumin; Methylglucamin; Methylglucamine; N-Methyl-D(−)-glucamine; N-Methyl-D-glucamine; N-Methylglucamine; N-Methylsorbitylamine; NSC 52907; NSC 7391. It also has the CA Index Name D-Glucitol, 1-deoxy-1-(methylamino)-(9Cl). A chemical formula for meglumine is as follows:

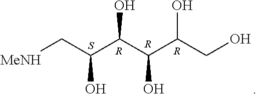

In one embodiment, the polyhydroxy amine compound comprises from about 0.1% to about 50% by weight of the composition. In further embodiments, the polyhydroxy amine compound comprises from about 0.25% to about 20% by weight of the composition. In yet further embodiments, the polyhydroxy amine compound comprises from about 0.5% to about 10% by weight of the composition. In yet further embodiments, the polyhydroxy amine compound comprises from about 1% to about 5% by weight of the composition.

vi. Chelating Agents

The compositions of the present invention can further comprise a chelating agent. The chelating agent is defined herein as excluding the cyclodextrin, the polyhydroxy amine compound, or any of the other components described herein, even though the cyclodextrin, the polyhydroxy amine compound, or other components described herein can also have chelating properties. An example of a chelating agent useful herein is EDTA, also known as ethylenediaminetetraacetic acid, or a salt thereof. Useful salts include, for example, a sodium salt, a potassium salt, a calcium salt, a magnesium salt, and mixtures of these salts. An example of a mixture of salts or a mixed salt is the monosodium monocalcium salt of EDTA. It is found that the disodium salt of EDTA, also known as disodium EDTA, is useful herein. For convenience, the disodium EDTA can first be separately prepared as an aqueous solution for use in formulating the compositions of the present invention.

In one embodiment, the disodium EDTA comprises from about 0.0010% to about 0.10% by weight of the composition. In further embodiments, the disodium EDTA comprises from about 0.0050% to about 0.050% by weight of the composition. In yet further embodiments, the disodium EDTA comprises from about 0.010% to about 0.020% by weight of the composition. In other embodiments the disodium EDTA comprises about 0.010% of the composition, or about 0.011% of the composition, or about 0.012% of the composition, or about 0.013% of the composition, or about 0.014% of the composition, or about 0.015% of the composition, or about 0.016% of the composition, or about 0.017% of the composition, or about 0.018% of the composition, or about 0.019% of the composition, or about 0.020% of the composition. These weight percentages of the disodium EDTA described herein are on the basis of the ethylenediaminetetraacetic acid.

vii. pH Modifiers and pH of the Compositions

The compositions of the present invention can further comprise various materials for modifying or adjusting the pH of the composition. Such materials include acids, bases, buffer systems, etc. Non-limiting examples of such pH modifiers include, for example, hydrochloric acid and sodium hydroxide. Examples of other useful materials include triethanolamine, sodium carbonate, and lysine. Furthermore, the polyhydroxy amine compound, such as described above, can be used as a pH modifier. More specifically, the polyhydroxy amine compound, meglumine, can be used as a pH modifier.

The compositions of the present invention should have a pH so that the composition is suitable for administration to a patient or subject. The compositions have a pH from about pH 7 to about pH 11. In further embodiments, the compositions have a pH from about pH 8 to about pH 10. In further embodiments, the compositions have a pH from about pH 8.5 to about pH 9.5. In further embodiments, the compositions have a pH from about pH 8.8 to about pH 9.2. In further embodiments, the compositions have a pH of about 9.0.

viii. Additional Components

Pharmaceutical compositions of the present invention comprise an effective amount of a quinolone carboxylic acid derivative or a pharmaceutically acceptable salt thereof and an aluminum compound, or one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains an effective amount of a quinolone carboxylic acid derivative or a pharmaceutically acceptable salt thereof and an aluminum compound will be known to those of skill in the art in light of the present disclosure, as exemplified by Remington's Pharmaceutical Sciences, 18th ed. Mack Printing Company, 1990, incorporated herein by reference. Moreover, for animal (e.g., human) administration, it will be understood that preparations should meet sterility, pyrogenicity, general safety and purity standards as required by the Food and Drug Administration's Office of Biological Standards.

The compositions of the present invention can further comprise one or more additional components selected from a wide variety of excipients known in the pharmaceutical formulation art. According to the desired properties of the tablet or capsule, any number of ingredients can be selected, alone or in combination, based upon their known uses in preparing the compositions of the present invention. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th ed. Mack Printing Company, 1990, pp. 1289-1329, the contents of which are incorporated herein by reference). Except insofar as any conventional carrier is incompatible with the active ingredient, its use in the therapeutic or pharmaceutical compositions is contemplated. Such ingredients include, but are not limited to solvents (e.g., ethanol), dispersion media, coatings (e.g., enteric polymers), isotonic agents, absorption delaying agents, salts, drugs, drug stabilizers, gels, plasticizers, binders, excipients, disintegration agents, lubricants, sweetening agents, flavoring agents, dyes, colorants; waxes, gelatin; preservatives (e.g., antibacterial agents and antifungal agents such as methyl paraben, sodium benzoate, and potassium benzoate); antioxidants (e.g., butylated hydroxyanisole (“BHA”), butylated hydroxytoluene (“BHT”), and vitamin E and vitamin E esters such as tocopherol acetate); surfactants; UV-absorbers, such like materials and combinations thereof. Exemplary cellulose ethers include hydroxyalkyl celluloses and carboxyalkyl celluloses. Exemplary cellulose ethers include hydroxyethyl celluloses, hydroxypropyl celluloses, hydroxypropylmethyl-celluloses, carboxy methylcelluloses, and mixtures thereof.

In some embodiments, the intravenous compositions of the present invention comprise a carrier. The carrier can be a dextrose solution or saline, at a pharmaceutically acceptable concentration. The intravenous composition comprising a carrier can be administered to a patient via an intravenous bag.

ix. Processing

The compositions for intravenous administration described herein are made using convention equipment and mixing techniques. Examples of techniques used to make the compositions described herein are provided in Remington's Pharmaceutical Sciences, 17th edition, 1985, Editor: Alfonso R. Gennaro, Mack Publishing Company, Easton, Pa. 18042, which is incorporated by reference herein.

Lyophilization, also known as freeze-drying is a dehydration process to remove the liquid, typically water and other relatively volatile solvents, from a material. Freeze drying works by freezing the material and then reducing the surrounding pressure and, as appropriate, adding enough heat to allow the frozen mobile water and other solvents in the material to sublime directly from the solid phase to gas.

x. Packaging

The compositions of the present invention can be packaged in standard, commercially available containers such as vials for liquid or lyophile storage. In some embodiments, the vial is glass. The glass can be colorless or colored, clear or amber. Various types of closure systems can be used such as screw vials (closed with screw cap), lip vials (closed with a stopper), or crimp vials (closed with a rubber stopper and a metal cap).

Additionally, the compositions of the present invention, including a reconstituted lyophile, can be further diluted into an intravenous delivery bag or bottle.

The invention encompasses kits that can simplify the administration of a quinolone carboxylic acid derivative or a composition comprising it to a subject. In one embodiment, a kit of the invention comprises a unit dosage form of a quinolone carboxylic acid derivative. In one embodiment the unit dosage form is a container, which can be sterile, containing an effective amount of a quinolone carboxylic acid derivative and a physiologically acceptable carrier or vehicle. Physiologically acceptable carriers include saline and dextrose solutions at pharmaceutically acceptable concentrations. Such compositions can be contained in an intravenous drip bag. The kit can further comprise a label or printed instructions instructing the use of the quinolone carboxylic acid derivative to treat, prevent, or reduce the risk of an infection. Kits of the invention can further comprise a device that is useful for administering the unit dosage forms. Examples of such a device include, but are not limited to, a bottle, a vial, a syringe and a drip bag. Other examples of devices include, but are not limited to, a patch, an inhaler, and an enema bag. In one embodiment, the device that is useful for administering the unit dosage forms is the container.

b. Compositions for Oral Administration

The compositions described herein comprise one or more of the following components.

i. Quinolone Carboxylic Acid Derivative

The orally administrable compositions of the present invention comprise a quinolone carboxylic acid derivative. This component is the same as described above under the intravenous compositions.

ii. Water

In one embodiment, the compositions of the present invention comprise from about 0.1% to about 99.9% water, in further embodiments from about 1% to about 99% water, in yet further embodiments from about 5% to about 95% water, and in yet further embodiments from about 10% to about 90% water. In defining a composition, the amount of water can be designated as “q.s.” or “Q.S.”, which means as much as suffices, to provide a final composition or volume of 100%.

iii. Sugars and Sugar Alcohols

The compositions of the present invention, when further made into a lyophile, can further comprise a sugar, a sugar alcohol, or mixtures thereof. Without being limited by theory, these sugars and sugar alcohols are believed to aid in the formation of the lyophile during the lyophilization process. Typically, the lyophile is made by drying the composition under appropriate conditions, such as, for example, by freeze drying. Non-limiting examples of sugars include mannose, sucrose, dextrose, and mixtures thereof. Non-limiting examples of sugar alcohols useful herein include sorbitol, mannitol, xylitol and mixtures thereof.

In one embodiment, the compositions comprise from about 0.1% to about 50% of a sugar or sugar alcohol.

iv. Polyhydroxy Amine Compound

In one embodiment, the compositions of the present invention comprise a polyhydroxy amine compound. The polyhydroxy amine compound is separate from and does not encompass the polyhydroxy compound of the compositions of the present invention. The polyhydroxy amine compound is generally a C₃-C₈ straight, branched, or cyclic compound having 2 or more hydroxy substituents, and at least one amine (either substituted or unsubstituted) substituent.

In further embodiments the polyhydroxy amine compound is meglumine. Meglumine corresponds to CAS Registry Number 6284-40-8 and is also known as meglumine, USP; 1-Deoxy-1-(methylamino)-D-glucitol; N-Methyl-D-glucamine; Glucitol, 1-deoxy-1-(methylamino)-, D-(8Cl); Sorbitol, 1-deoxy-1-methylamino-(6Cl); 1-Deoxy-1-(methylamino)-D-glucitol; 1-Deoxy-1-methylaminosorbitol; D-(−)-N-Methylglucamine; Meglumin; Methylglucamin; Methylglucamine; N-Methyl-D(−)-glucamine; N-Methyl-D-glucamine; N-Methylglucamine; N-Methylsorbitylamine; NSC 52907; NSC 7391. It also has the CA Index Name D-Glucitol, 1-deoxy-1-(methylamino)-(9Cl). A chemical formula for meglumine is as follows:

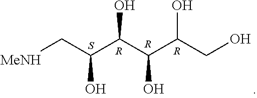

In one embodiment, the polyhydroxy amine compound comprises from about 0.1% to about 50% by weight of the composition. In further embodiments, the polyhydroxy amine compound comprises from about 0.25% to about 20% by weight of the composition. In yet further embodiments, the polyhydroxy amine compound comprises from about 0.5% to about 10% by weight of the composition. In yet further embodiments, the polyhydroxy amine compound comprises from about 1% to about 5% by weight of the composition.

v. Chelating Agents

The compositions of the present invention can further comprise a chelating agent. The chelating agent is defined herein as excluding the cyclodextrin, the polyhydroxy amine compound, or any of the other components described herein, even though the cyclodextrin, the polyhydroxy amine compound, or other components described herein can also have chelating properties. An example of a chelating agent useful herein is EDTA, also known as ethylenediaminetetraacetic acid, or a salt thereof. Useful salts include, for example, a sodium salt, a potassium salt, a calcium salt, a magnesium salt, and mixtures of these salts. An example of a mixture of salts or a mixed salt is the monosodium monocalcium salt of EDTA. It is found that the disodium salt of EDTA, also known as disodium EDTA, is useful herein. For convenience, the disodium EDTA can first be separately prepared as an aqueous solution for use in formulating the compositions of the present invention.

In one embodiment, the disodium EDTA comprises from about 0.0010% to about 0.10% by weight of the composition. In further embodiments, the disodium EDTA comprises from about 0.0050% to about 0.050% by weight of the composition. In yet further embodiments, the disodium EDTA comprises from about 0.010% to about 0.020% by weight of the composition. In other embodiments the disodium EDTA comprises about 0.010% of the composition, or about 0.011% of the composition, or about 0.012% of the composition, or about 0.013% of the composition, or about 0.014% of the composition, or about 0.015% of the composition, or about 0.016% of the composition, or about 0.017% of the composition, or about 0.018% of the composition, or about 0.019% of the composition, or about 0.020% of the composition. These weight percentages of the disodium EDTA described herein are on the basis of the ethylenediaminetetraacetic acid.

vi. pH Modifiers and pH of the Compositions

The compositions of the present invention can further comprise various materials for modifying or adjusting the pH of the composition. Such materials include acids, bases, buffer systems, etc. Non-limiting examples of such pH modifiers include, for example, hydrochloric acid and sodium hydroxide. Examples of other useful materials include triethanolamine, sodium carbonate, and lysine. Furthermore, the polyhydroxy amine compound, such as described above, can be used as a pH modifier. More specifically, the polyhydroxy amine compound, meglumine, can be used as a pH modifier.

The compositions of the present invention should have a pH so that the composition is suitable for administration to a patient or subject. The compositions have a pH from about pH 7 to about pH 11. In further embodiments, the compositions have a pH from about pH 8 to about pH 10. In further embodiments, the compositions have a pH from about pH 8.5 to about pH 9.5. In further embodiments, the compositions have a pH from about pH 8.8 to about pH 9.2. In further embodiments, the compositions have a pH of about 9.0.

vii. Additional Components

Pharmaceutical compositions of the present invention comprise an effective amount of a quinolone carboxylic acid derivative or a pharmaceutically acceptable salt thereof and an aluminum compound, or one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains an effective amount of a quinolone carboxylic acid derivative or a pharmaceutically acceptable salt thereof and an aluminum compound will be known to those of skill in the art in light of the present disclosure, as exemplified by Remington's Pharmaceutical Sciences, 18th ed. Mack Printing Company, 1990, incorporated herein by reference. Moreover, for animal (e.g., human) administration, it will be understood that preparations should meet sterility, pyrogenicity, general safety and purity standards as required by the Food and Drug Administration's Office of Biological Standards.

The compositions of the present invention can further comprise one or more additional components selected from a wide variety of excipients known in the pharmaceutical formulation art. According to the desired properties of the tablet or capsule, any number of ingredients can be selected, alone or in combination, based upon their known uses in preparing the compositions of the present invention. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th ed. Mack Printing Company, 1990, pp. 1289-1329, the contents of which are incorporated herein by reference). Except insofar as any conventional carrier is incompatible with the active ingredient, its use in the therapeutic or pharmaceutical compositions is contemplated. Such ingredients include, but are not limited to solvents (e.g., ethanol), dispersion media, coatings (e.g., enteric polymers), isotonic agents, absorption delaying agents, salts, drugs, drug stabilizers, gels, plasticizers, binders, excipients, disintegration agents, lubricants, sweetening agents, flavoring agents, dyes, colorants; waxes, gelatin; preservatives (e.g., antibacterial agents and antifungal agents such as methyl paraben, sodium benzoate, and potassium benzoate); antioxidants (e.g., butylated hydroxyanisole (“BHA”), butylated hydroxytoluene (“BHT”), and vitamin E and vitamin E esters such as tocopherol acetate); surfactants; UV-absorbers, such like materials and combinations thereof. Exemplary cellulose ethers include hydroxyalkyl celluloses and carboxyalkyl celluloses. Exemplary cellulose ethers include hydroxyethyl celluloses, hydroxypropyl celluloses, hydroxypropylmethyl-celluloses, carboxy methylcelluloses, and mixtures thereof.

Any of the excipients can be present in the oral formulation in an amount of about 0 mg to about 5 mg, about 5 mg to about 10 mg, about 10 mg to about 20 mg, about 20 mg to about 50 mg, about 50 mg to about 100 mg, about 100 mg to about 200 mg, about 200 mg to about 500 mg, about 500 mg to about 800 mg, or about 800 mg to about 1000 mg.

In some embodiments, microcrystalline cellulose is present in the oral formulation in an amount of about 0 mg to about 200 mg or about 200 mg to about 500 mg.

In some embodiments, water is present in the oral formulation in an amount of about 0 mg to about 200 mg.

In some embodiments, a binder is present in the oral formulation in an amount of about 0 mg to about 100 mg or about 0 to about 200 mg. In some embodiments, a disintegrant is present in the formulation in an amount of about 0 mg to about 100 mg or about 0 mg to about 200 mg.

In some embodiments, an enteric coating is present in a tablet in an amount of about 0 mg to about 20 mg or about 0 mg to about 200 mg.

In some embodiments, a plasticizer is present in a tablet in an amount of about 1 mg to about 30 mg.

viii. Processing

The compositions for oral administration described herein are made using conventional equipment and mixing techniques, such as dry granulation or wet granulation. Examples of techniques used to make the compositions described herein are provided in Remington's Pharmaceutical Sciences, 17th edition, 1985, Editor: Alfonso R. Gennaro, Mack Publishing Company, Easton, Pa. 18042, which is incorporated by reference herein.

4. Gastrointestinal Tolerability

The compositions of the present invention have improved gastrointestinal tolerability. Also, the compositions of the present invention reduce the potential for gastrointestinal side effects. Non-limiting gastrointestinal side effects include diarrhea, flatulence, nausea, vomiting, abdominal pain, dyspepsia, belching, bloating, gastritis, and general abdominal discomfort. The gastrointestinal tolerability of the compositions of the present invention can be evaluated using various in vitro and in vivo models are described in the Examples below.

5. Doses and Methods of Treating, Preventing, or Reducing the Risk of Infections

In some embodiments, the compositions disclosed herein are useful in treating microbial infections (e.g., bacterial infections).

The compositions of the present invention are useful for treating, preventing or reducing the risk of bacterial infection, e.g., a skin infection, a skin and skin structure infections such as a complicated skin and skin structure infection (cSSSI), an uncomplicated skin and skin structure infection (uSSSI) and an acute bacterial skin and skin structure infection (ABSSSI), pneumonia and other respiratory tract infections such as a community respiratory-tract infection, a nosocomial (hospital-acquired) pneumonia, a community acquired pneumonia, a hospital acquired community pneumonia and a post-viral pneumonia, an abdominal infection, a urinary tract infection, bacteremia, septicemia, endocarditis, an atrio-ventricular shunt infection, a vascular access infection, meningitis, an infection due to surgical or invasive medical procedures, a peritoneal infection, a bone infection, a joint infection, a methicillin-resistant Staphylococcus aureus (MRSA) infection, a vancomycin-resistant Enterococci infection, a linezolid-resistant organism infection, tuberculosis, a quinolone resistant Gram-positive infection, a ciprofloxacin resistant methicillin resistant (MRSA) infection, bronchitis, uncomplicated gonorrhea, and a multi-drug resistant (MDR) Gram-negative infection.

The dose of active compound and mode of administration, e.g., injection, intravenous drip, etc. will depend upon the intended patient or subject and the targeted microorganism, e.g., the target bacterial organism. Dosing strategies are disclosed in L. S. Goodman, et al., The Pharmacological Basis of Therapeutics, 201-26 (5th ed. 1975), the entire contents of which are herein incorporated in its entirety.

Compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals.

Compositions of the present invention are made in the form of a dry mixture, an aqueous solution, an aqueous suspension, a non-aqueous solution, a non-aqueous suspension, or an emulsion. The pharmaceutical compositions are made in unit dosage forms, which may further be in the form of a tablet or a capsule. The tablet may be a bilayer tablet comprising more than one layer. In one embodiment, the bilayer tablet comprises a first and a second layer. In some embodiments, the first layer comprises the quinolone carboxylic acid derivative or a pharmaceutically acceptable salt or ester thereof and the second layer comprises the aluminum compound. In some embodiments, the second layer in the bilayer tablet is a delayed or sustained or controlled release layer, wherein the second layer is released in the gastrointestinal tract downstream from the stomach. Alternatively, the quinolone carboxylic acid derivative or a pharmaceutically acceptable salt or ester thereof and the aluminum compound are administered as separate unit dosage forms. In some embodiments, when the quinolone carboxylic acid derivative or a pharmaceutically acceptable salt or ester thereof and the aluminum compound are administered as separate unit dosage forms, the quinolone carboxylic acid derivative or a pharmaceutically acceptable salt or ester thereof is administered first and the aluminum compound is administered second and within two hours of the quinolone carboxylic acid derivative. In yet other embodiments, the quinolone carboxylic acid derivative or a pharmaceutically acceptable salt or ester and the aluminum compound are concurrently administered as separate unit dosage forms.

In conjunction with the methods of the present invention, pharmacogenomics (i.e. the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a drug as well as tailoring the dosage and/or therapeutic regimen of treatment with the drug.

Generally, an effective amount of dosage of active compound will be in the range of from about 0.1 to about 100 mg/kg of body weight/day. In one embodiment, the amount will be from about 1.0 to about 50 mg/kg of body weight/day. The amount administered will also likely depend on such variables as the overall health status of the patient, the relative biological efficacy of the compound delivered, the formulation of the drug, the presence and types of excipients in the formulation, the route of administration, and the infection to be treated, prevented, or reducing the risk of Also, it is to be understood that the initial dosage administered can be increased beyond the above upper level in order to rapidly achieve the desired blood-level or tissue level, or the initial dosage can be smaller than the optimum.

In some embodiments, the compositions disclosed herein comprise a dose of active compound of about 0.1 to about 1500 mg per dose. In some embodiments, the compositions disclosed herein comprise a dose of active compound of about 100 mg to about 750 mg per dose. In some embodiments, the compositions disclosed herein comprise a dose of active compound of about 250 mg to about 500 mg per dose. In some embodiments, the compositions disclosed herein comprise a dose of active compound of about 300 mg per dose. In some embodiments, the compositions disclosed herein comprise a dose of active compound of about 400 mg per dose. In some embodiments, the compositions disclosed herein comprise a dose of active compound of about 450 mg per dose. In some embodiments, the compositions disclosed herein comprise a dose of active compound of about 0.1 to about 10 mg per dose, about 10 mg to about 20 mg per dose, about 20 mg to about 50 mg per dose, about 50 mg to about 100 mg per dose, about 100 mg to about 200 mg per dose, about 200 mg to about 500 mg per dose, about 500 mg to about 1000 mg per dose or about 1000 mg to about 1500 mg per dose. Non-limiting examples of doses, which can be formulated as a unit dose for convenient administration to a patient include: about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, and about 1500 mg. The foregoing doses are useful for administering the compounds of the present invention according to the methods of the present invention. The foregoing doses are particularly useful for administering the quinolone carboxylic acid derivatives of the present invention, particularly the compound known by the name delafloxacin and pharmaceutically acceptable salts, esters and prodrugs thereof.

As is understood by one of ordinary skill in the art, generally, when dosages are described for a pharmaceutical active, the dosage is given on the basis of the parent or active moiety. Therefore, if a salt, hydrate, or another form of the parent or active moiety is used, a corresponding adjustment in the weight of the compound is made, although the dose is still referred to on the basis of the parent or active moiety delivered. As a non-limiting example, if the parent or active moiety of interest is a monocarboxylic acid having a molecular weight of 250, and if the monosodium salt of the acid is desired to be delivered at the same dosage, then an adjustment is made recognizing that the monosodium salt would have a molecular weight of approximately 272 (i.e. minus 1H or 1.008 atomic mass units and plus 1 Na or 22.99 atomic mass units). Therefore, a 250 mg dosage of the parent or active compound would correspond to about 272 mg of the monosodium salt, which would also deliver 250 mg of the parent or active compound. Said another way, about 272 mg of the monosodium salt would be equivalent to a 250 mg dosage of the parent or active compound.

In one embodiment, compositions of the invention is useful in the manufacture of a medicament for treating, preventing or reducing the risk of infection in a patient in need thereof. In another embodiment, delafloxacin, or a pharmaceutically acceptable salt thereof, is useful in the manufacture of a medicament for treating, preventing or reducing the risk of infection in a patient in need thereof. Such infections can be due to, e.g., a skin infection, a skin and skin structure infections such as a complicated skin and skin structure infection (cSSSI), an uncomplicated skin and skin structure infection (uSSSI) and an acute bacterial skin and skin structure infection (ABSSSI), pneumonia and other respiratory tract infections such as a community respiratory-tract infection, a nosocomial (hospital-acquired) pneumonia, a community acquired pneumonia, a hospital acquired community pneumonia and a post-viral pneumonia, an abdominal infection, a urinary tract infection, bacteremia, septicemia, endocarditis, an atrio-ventricular shunt infection, a vascular access infection, meningitis, an infection due to surgical or invasive medical procedures, a peritoneal infection, a bone infection, a joint infection, a methicillin-resistant Staphylococcus aureus (MRSA) infection, a vancomycin-resistant Enterococci infection, a linezolid-resistant organism infection, tuberculosis, a quinolone resistant Gram-positive infection, a ciprofloxacin resistant methicillin resistant (MRSA) infection, bronchitis, uncomplicated gonorrhea, and a multi-drug resistant (MDR) Gram-negative infection.

Using delafloxacin as a non-limiting example, an example of a composition useful in the methods of the present invention can contain about 300 mg of delafloxacin, or a pharmaceutically acceptable salt thereof.

### 6. EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Ingredients are identified by chemical, USP, or CTFA name.

The following formulations are preparing using mixing techniques and equipment familiar to one of ordinary skill in the art.

These formulations are useful for oral administration to a patient for treating, preventing, or reducing the risk of a microbial infection, e.g., a skin infection, including uncomplicated skin infections, skin and soft tissue infections, complicated skin infections, pneumonia, including e.g., community acquired pneumonia, nosocomial (hospital acquired) pneumonia, hospital acquired community pneumonia, post-viral pneumonia, an abdominal infection, a urinary tract infection, bacteremia, septicemia, endocarditis, an atrio-ventricular shunt infection, a vascular access infection, meningitis, infection due to surgical or invasive medical procedures, a peritoneal infection, a bone infection, a joint infection, a methicillin-resistant Staphylococcus aureus infection, a vancomycin-resistant Enterococci infection, a linezolid-resistant organism infection, and tuberculosis. More specifically, this formulation is useful for reducing the risk of or preventing infection due to a surgical or invasive medical procedure to be performed upon the patient, and in such case, the formulation can be administered just prior to or up to about 1 hour prior to the surgical or invasive medical procedure.

Examples 1-2, below, illustrate compositions for intravenous use which are useful as part of an intravenous/oral administration regimen for a patient for treating, preventing, or reducing the risk of a microbial infection.

Example 3, below, illustrates an oral composition useful as part of an intravenous/oral administration regimen for a patient for treating, preventing, or reducing the risk of a microbial infection.

Example 4, below, illustrates an intravenous/oral administration regimen for a patient for treating, preventing, or reducing the risk of a microbial infection.

### Example 1

### Formulation for Intravenous Administration

The following is a sample formulation comprising a liquid formulation of delafloxacin. Table 1 provides a Quantitative Composition of 25 mg/mL Delafloxacin Liquid Formulation.

TABLE 1

| | | | % w/w in |
|---|---|---|---|
| | | | lyophilized |
| Ingredient | mg/mL | mg/dose | powder |
| Delafloxacin Meglumine | 25.000 | 300¹ | 14.83 |
| (amount as free acid) | | | |
| Meglumine (anhydrous, | 4.88 | 58.6 | 2.03 |
| mw 195.21) | | | |
| Betadex Sulfobutyl Ether | 200 | 2400 | 83.0 |
| Sodium (Captisol ®) (also | | | |
| referred to as beta-cyclodextrin | | | |
| sulfobutyl ether sodium) | | | |
| Edetate disodium (Disodium | 0.28 | 3.4 | 0.12 |
| EDTA) | | | |
| Sterile Water for Injection (WFI) | q.s. | q.s. | |
| 1N Sodium hydroxide solution² | q.s. | q.s. | |
| 1N Hydrochloride solution² | q.s. | q.s. | |
| Total | 1090 | | 100 |
| Density | 1.087 g/mL | | |
| Final pH | 9.0 (±0.1) | | |

¹The amount of delafloxacin is based on a theoretical potency of 100% as free acid. The exact amount will vary depending on the purity of delafloxacin as shown in the equation: Delafloxacin added = quantity of free acid (g) × (100/purity %) × (100/69.30 (salt value)) × (100/(100-water %-IPA %)). Conversion factor between delafloxacin salt to free acid = 1.4429. Assuming 100% pure, the theoretical amount of drug substance is 428.7 mg (eq. to 300 mg free acid).
²1N sodium hydroxide solution and/or 1N hydrochloric acid solution may be used if necessary to adjust the final solution pH to 9.0 ± 0.1.

### Procedure:

1. Add the required amount of beta-cyclodextrin sulfobutyl ether sodium (Captisol®) to the tank with 70-75% of the total amount of water for injection and mix until dissolved.
2. Add the required amount of meglumine to the solution and mix until dissolved.
3. Add the required amount of edetate disodium (EDTA) and mix until dissolved.
4. Test the solution for pH. The target pH is 9.7 (±0.1). Adjust with hydrochloric acid (as a 1N solution) to pH 9.7 (±0.1). In case the pH is adjusted below 9.6, sodium hydroxide (as a 1N solution) can be used to further adjust the pH.
5. Add the required amount of delafloxacin meglumine (RX-3341-meglumine), corrected for purity, water and salt content, and mix until dissolved.
6. Test for pH. The target pH is 9.0 (·0.1).
7. q.s. to the final weight or volume with Water for Injection.
8. Sterile filter solution (two filters 0.22 um) and fill into vials. *In further formulations, the amount of EDTA solution add is increased to 0.15 mg/mL.

The foregoing composition is useful for intravenous administration to a patient for treating, preventing, or reducing the risk of a microbial infection.

Based on the foregoing formulation in Table 1, the following mg of the indicated component is delivered in a given dosage as shown in Table 2.

TABLE 2

| | 100 mg strength | | 300 mg strength | | 500 mg strength | |
|---|---|---|---|---|---|---|
| | dosage | | dosage | | dosage | |
| Volume of | 4 | mL | 12 | mL | 20 | mL |
| Dosage | | | | | | |
| Delafloxacin | 100 | mg | 300 | mg | 500 | mg |
| Meglumine | 19.52 | mg | 58.56 | mg | 97.6 | mg |
| Captisol | 800 | mg | 2400 | mg | 4000 | mg |
| Disodium | 0.44 | mg | 1.32 | mg | 2.2 | mg |
| EDTA | | | | | | |

The foregoing intravenous composition is useful as part of an intravenous/oral administration regimen for a patient for treating, preventing, or reducing the risk of a microbial infection.

### Example 2

### Lyophilisates for Reconstitution for Intravenous Administration

Formulations can also be prepared as lyophilisates. For example, the formulation of Example 1, above can also be prepared as a lyophile. This is accomplished by sterile filtering the solutions into sterile vials suitable for lyophilization, and then freeze-drying the vials using conventional freeze-drying techniques.

Such formulations are reconstituted with water or another appropriate aqueous based solution. These lyophilisates are a compact and convenient form to store the formulation.

The foregoing lyophilized intravenous composition is useful as part of an intravenous/oral administration regimen for a patient for treating, preventing, or reducing the risk of a microbial infection.

### Example 3

### First Formulation for Oral Administration

The following is a sample formulation comprising the combination of delafloxacin and an effervescent agent. The bilayer tablet of this example was made using standard formulation and tableting techniques. Table 3 provides a Quantitative Composition of 400 mg Delafloxacin Bilayer Tablet.

TABLE 3

| Ingredient | mg/tablet | % w/w |
|---|---|---|
| Drug Layer, Intra-granular (wet granulation) | | |
| Delafloxacin (Meglumine salt)⁽¹⁾ | 577.2 | 41.23 |
| Cellulose, Microcrystalline, Avicel PH 101⁽²⁾ | 287.8 | 20.56 |
| Povidone K29/32 | 30. | 2.1 |
| Crospovidone, NF (Kollidon CL) | 50. | 3.6 |
| Water, USP ⁽³⁾ | 170. | N/A |
| Water, USP ⁽⁴⁾ | q.s. | N/A |
| | (Up to 100) | |
| Drug Layer, Extra-granular | | |
| Crospovidone, NF (Kollidon CL) | 50. | 3.6 |
| Magnesium stearate, NF | 5.0 | 0.36 |
| Drug Layer Total | 1000 | 71.5 |
| Buffering Layer, Intra-granular (Dry granulation) | | |
| Sodium bicarbonate, powder, USP | 140. | 10.0 |
| Sodium phosphate monobasic, monohydrate, USP | 5.5 | 0.39 |
| Citric acid, Anhydrous, powder, USP | 5.5 | 0.39 |
| Cellulose, Microcrystalline (Avicel PH 101) | 247 | 17.6 |
| Magnesium stearate, NF | 0.8 | 0.06 |
| Buffering Layer, Extra-granular | | |
| Magnesium stearate, NF | 1.2 | 0.086 |
| Buffering Layer Total | 400 | 28.5 |
| Total for Bilayer Tablet | 1400 | 100 |

⁽¹⁾Equivalent to 400 mg free acid (based on 69.3% salt conversion). The actual values may vary slightly depending on the purity and residue solvent content from the certificate of analysis (CoA).
⁽²⁾The exact amount will be adjusted based on the purity of API.
⁽³⁾ For binder solution (PVP solution) preparation, and granulation. Removed during drying process.
⁽⁴⁾ Maximum additional water for granulation, removed during drying process.

Table 4 provides a simplified Quantitative Composition of 400 mg Delafloxacin Bilayer Tablet, showing total amounts of each ingredient (i.e. where the components are not separated by granulation or layer).

TABLE 4

| Ingredient | mg/tablet | % w/w |
|---|---|---|
| Delafloxacin (Meglumine salt)⁽¹⁾ | 577.2 | 41.23 |
| Cellulose, Microcrystalline ⁽²⁾ | 535 | 38.2 |
| Povidone K30 | 30. | 2.1 |
| Crospovidone, NF | 100. | 7.14 |
| Sodium bicarbonate | 140. | 10.0 |
| Sodium phosphate monobasic, monohydrate | 5.5 | 0.39 |
| Citric acid, Anhydrous | 5.5 | 0.39 |
| Magnesium stearate | 7.0 | 0.50 |
| Total | 1400 | 100 |

⁽¹⁾Equivalent to 400 mg free acid (based on 69.3% salt conversion). The actual values may vary slightly depending on the purity and residue solvent content from the certificate of analysis (CoA).
⁽²⁾ The exact amount will be adjusted based on the purity of API.

The foregoing composition is useful for oral administration to a patient for treating, preventing, or reducing the risk of a microbial infection.

### Example 4

### Second Formulation for Oral Administration

The following is a sample formulation comprising the combination of delafloxacin and an effervescent agent. The single layer tablet of this example was made using standard formulation and tableting techniques. Table 5 provides a Quantitative Composition of 450 mg Delafloxacin Single Layer Tablet.

TABLE 5

| | mg/tablet | |
|---|---|---|
| Ingredient | (450 mg) | % w/w |
| Drug Granulation | | |
| Delafloxacin (Meglumine salt)⁽¹⁾ | 649.4 | 47.40 |
| Microcrystalline Cellulose, PH 101⁽²⁾ | 295.8 | 21.59 |
| Povidone 30 LP | 33.8 | 2.47 |
| Crospovidone | 84.0 | 6.13 |
| Water, USP ⁽³⁾ | 191.3 | NA |
| Water, USP ⁽⁴⁾ | 60. | NA |
| Total drug granule | 1063 | 77.6 |
| Extragranular | | |
| Sodium bicarbonate | 140. | 10.2 |
| Sodium phosphate monobasic, monohydrate | 5.5 | 0.40 |
| Citric acid, Anhydrous | 5.5 | 0.40 |
| Crospovidone | 25.0 | 1.82 |
| Microcrystalline Cellulose, PH 101 | 121.0 | 8.83 |
| Magnesium stearate | 10. | 0.73 |
| Total extragranular | 307 | 22.4 |
| FINAL TABLET | 1370 | 100 |

⁽¹⁾The amount of delafloxacin is based on a theoretical potency of 100% as free acid (based on 69.3% salt conversion). The exact amount may vary slightly depending on the purity and residue solvent content from the certificate of analysis (CoA).
⁽²⁾The exact amount of microcrystalline cellulose will be adjusted based on the purity of API.
⁽³⁾ For binder solution preparation, and granulation. Removed during drying process.
⁽⁴⁾ Target additional water for granulation, final amount depending on granulation end point with upper limit of 100 mg. This water is removed during drying process.

The foregoing composition is useful for oral administration to a patient for treating, preventing, or reducing the risk of a microbial infection. In particular, this composition was successful at achieving an AUC nearly identical to that of a 300 mg intravenous formulation of delafloxacin meglumine in clinical trials, as discussed in Example 6.

Table 6 provides a simplified Quantitative Composition of 450 mg Delafloxacin Single Layer Tablet, showing total amounts of each ingredient (i.e. where the components are not separated by granulation).

TABLE 6

| Ingredient | mg/tablet | % w/w |
|---|---|---|
| Delafloxacin (Meglumine salt)⁽¹⁾ | 649.4 | 47.40 |
| Microcrystalline Cellulose, PH 101 ⁽²⁾ | 416.8 | 30.42 |
| Povidone 30 LP | 33.8 | 2.47 |
| Crospovidone | 109 | 7.96 |
| Sodium bicarbonate | 140. | 10.2 |
| Sodium phosphate monobasic, monohydrate | 5.5 | 0.40 |
| Citric acid, Anhydrous | 5.5 | 0.40 |
| Magnesium stearate | 10. | 0.73 |
| Total | 1370 | 100 |

⁽¹⁾The amount of delafloxacin is based on a theoretical potency of 100% as free acid (based on 69.3% salt conversion). The exact amount may vary slightly depending on the purity and residue solvent content from the certificate of analysis (CoA).
⁽²⁾ The exact amount of microcrystalline cellulose will be adjusted based on the purity of API.

### Example 5

### First Dosing Regimen with Intravenous and Oral Formulations

Compositions of the present invention were administered to human subjects in a clinical trial. The subjects were administered an intravenous formulation containing delafloxacin meglumine API (300 mg per intravenous dosage on a free acid basis) described in Example 1 for three days followed by four days of twice-daily oral administration of the formulation described in Example 3, (4) capsules containing delafloxacin meglumine API (100 mg delafloxacin per capsule on a free acid basis for a total of 400 mg delafloxacin for the four capsules on a free acid basis). The delafloxacin meglumine capsules were prepared by weighing the delafloxacin meglumine, 100 mg on a delafloxacin free acid basis, into commercially available gray, size zero, gelatin capsules, which were then sealed.

The following dosing regimen was followed in this nine (9) day clinical trial. On days 1 and 2, the subjects were administered placebo twice daily (BID). On days 3 through 5, the subjects were co-administered an intravenous formulation containing the delafloxacin meglumine once per day as a one-hour infusion. On days 6 through 9, the subjects were administered the capsules containing the delafloxacin meglumine twice daily, twelve hours apart, under fasted conditions. The formulations presented below will be used. The delafloxacin amount is presented as free acid. Unless specified, delafloxacin meglumine salt is used in the formulation. The oral dose is 400 mg and intravenous dose is 300 mg, amount as free acid.

Various evaluations and assessments were made during the course of the study including drug blood levels for determining pharmacokinetic/pharmacodynamic (PK/PD) parameters, incidence of diarrhea (both self-reported and clinical assessment using the standard Bristol stool chart), and adverse event reporting.

The administration of the delafloxacin meglumine intravenous formulation followed by the administration of the delafloxacin meglumine oral formulation was found to be well tolerated, and provided for reduced side effects as compared to all-oral formulations. The foregoing compositions and methods are useful for administration to a patient for treating, preventing, or reducing the risk of a microbial infection.

In clinical trials, the most frequently reported Treatment-Emergent Adverse Events (TEAEs) overall were classified as gastrointestinal disorders (13.3%) with diarrhea the most commonly reported TEAE (10.0%) followed by abdominal pain (3.3%) as shown in Table 7. The highest percentage of subjects who reported diarrhea received oral delafloxacin meglumine salt capsules or oral delafloxacin potassium salt capsules (15.0% each). Abdominal pain, the second most commonly reported TEAE, was reported by subjects who received oral delafloxacin potassium salt capsules (15.0%). No side effect was reported in the group with intravenous administration followed by oral treatment.

TABLE 7
GI Adverse Events Reported by Two or
More Subjects (Safety Population)

| | | | Meglumine | |
|---|---|---|---|---|
| | | | Salt 300 mg | |
| | Meglumine | Potassium | IV then 400 mg | |
| | Salt (A¹) | Salt (B¹) | oral (C¹) | Overall |
| | n = 20 | n = 20 | n = 20 | n = 60 |
| Gastrointestinal | 5 (25.0%) | 3 (15.0%) | 0 | 8 (13.3%) |
| disorders | | | | |
| Diarrhea | 3 (15.0%) | 3 (15.0%) | 0 | 6 (10.0%) |
| Abdominal Pain | 0 | 2 (10.0%) | 0 | 2 (3.3%) |
| Nausea | 0 | 1 (5.0%) | 0 | 1 (1.7%) |

¹Regimen A: 400-mg delafloxacin meglumine salt (4 × 100-mg capsules)
Regimen B: 400-mg delafloxacin potassium salt (4 × 100-mg capsules)
Regimen C: 300-mg delafloxacin meglumine salt infused intravenously followed by 400-mg oral delafloxacin meglumine salt (4 × 100-mg capsules)

### Example 6

### Pharmacokinetics and Relative Bioavailability of Intravenous and Oral Formulations

The pharmacokinetics and relative bioavailabilities of a 300 mg delafloxacin intravenous dosage form according to Example 1 and a 450 mg dose of oral delafloxacin according to Example 4 were measured in a Phase 1, single-dose, open-label, randomized, 2-period, 2-sequence crossover study. 56 healthy subjects received a single 450-mg dose of oral delafloxacin and a single 300-mg delafloxacin IV infusion in 1 of 2 treatment sequences. Plasma samples were analyzed for delafloxacin concentrations with a validated LC-MS/MS method. Pharmacokinetic parameters were calculated using noncompartmental methods. The results are shown below in Table 8.

TABLE 8
Results of AUC Exposure and Cₘₐₓ

| | | | | Ratio | |
|---|---|---|---|---|---|
| | | | | (%) of | |
| | | | | Geometric | 90% CI of |
| Parameter | | | Geometric | LS Means | the Ratio |
| (unit) | Treatment | N | LS Mean | (Test/Ref) | (Test/Ref) |
| AUC_{0-inf} | 450 mg Tablet | 42 | 23.0 | 87.7 | 83.6, 92.0 |
| (μg · h/mL) | 300 mg Infusion | 49 | 26.2 | | |
| AUC₀₋ₜ | 450 mg Tablet | 55 | 22.2 | 84.4 | 80.9, 88.2 |
| (μg · h/mL) | 300 mg Infusion | 55 | 26.3 | | |
| Cₘₐₓ | 450 mg Tablet | 55 | 5.80 | 55.2 | 51.5, 59.1 |
| (μg/mL) | 300 mg Infusion | 55 | 10.5 | | |

To assess the relative exposures of the oral form (Test) to the IV form (Reference), a linear mixed-effect model was performed on the natural logarithm (ln)-transformed values of AUC₀₋ₜ, AUC_{0-inf}, and Cₘₐₓ with sequence, treatment, and period as fixed effects and subject nested within sequence as a random effect.

Equivalence in AUC exposure between the oral and IV forms was concluded since the 90% CIs for the Test-to-Reference ratios of geometric means were entirely contained within the predefined criterion interval of 80% to 125% for AUC₀₋ₜ and AUC_{0-inf}. Equivalence for Cₘₐₓ was not concluded.

16 treatment-emergent AEs (TEAE) were reported, the most frequent of which were headache (5.4%), followed by diarrhea (3.6%). 2 TEAEs were considered moderate in severity, but the remainders were considered mild, and none led to study discontinuation.

### Example 7

### Second Dosing Regimen with Intravenous and Oral Formulations

An additional clinical study protocol was designed to administer compositions of the present invention to human subjects with ABSSSI. Subjects with a creatinine clearance (“CrCl”) greater than 29 mL/min at screening will be administered an intravenous formulation containing delafloxacin meglumine API (1 hour intravenous infusion of 300 mg on a free acid basis) described in Example 1 every 12 hours for six doses (i.e., three days) followed by four to 22 twice-daily oral administration of the single layer tablet formulation described in Example 4 (450 mg on a free acid basis). Subjects with a creatinine clearance (“CrCl”) of 15 to 19 mL/min at screening were administered an intravenous formulation containing delafloxacin meglumine API (1 hour intravenous infusion of 200 mg on a free acid basis) described in Example 1 every 12 hours for 10 to 28 doses based on the investigator's judgment.

### INCORPORATION BY REFERENCE

The entire disclosure of each of the patent documents, including certificates of correction, patent application documents, scientific articles, governmental reports, websites, and other references referred to herein is incorporated by reference in its entirety for all purposes.

### EQUIVALENTS

The invention can be embodied in other specific forms without departing from the spirit or essential characteristics thereof. The foregoing embodiments are therefore to be considered in all respects illustrative rather than limiting on the invention described herein.

What is claimed is:

1. A method for treating or reducing the risk of a microbial infection in a patient in need thereof comprising:
(a) intravenously administering an intravenous formulation comprising a pharmaceutically effective amount of a quinolone carboxylic acid derivative or a pharmaceutically acceptable salt or ester thereof according to a schedule of once daily or twice daily from about 1 to about 7 days consecutively; and thereafter
(b) orally administering an oral formulation comprising a pharmaceutically effective amount of said quinolone carboxylic acid derivative or said pharmaceutically acceptable salt or ester thereof according to a schedule of once daily or twice daily from about 1 to about 14 days consecutively;
wherein said quinolone carboxylic acid derivative corresponds to the following compound (A) 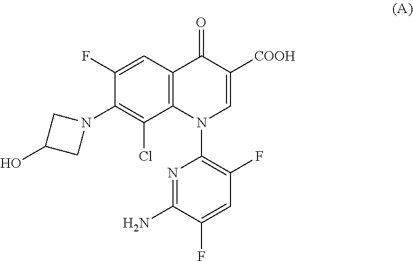 [SMILES: Nc1nc(-n2cc(C(=O)O)c(=O)c3cc(F)c(N4CC(O)C4)c(Cl)c32)c(F)cc1F]
or a pharmaceutically acceptable salt or ester thereof.

2. The method of claim 1, wherein
(a) said intravenous administration is from about 1 to about 4 days consecutively; and
(b) said oral administration is from about 1 to about 7 days consecutively.

3. The method of claim 1, wherein said intravenous administration is from about 1 to about 3 days consecutively.

4. The method of claim 1, wherein said oral administration is from about 2 to about 11 days consecutively.

5. The method of claim 1, wherein said oral administration is from about 1 to about 4 days consecutively.

6. The method of claim 1, wherein said oral administration is twice daily.

7. The method of claim 1, wherein said pharmaceutically acceptable salt of said quinolone carboxylic acid derivative is a D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate.

8. The method of claim 1, wherein said pharmaceutically acceptable salt of said quinolone carboxylic acid derivative is a crystalline D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate characterized by an X-ray powder diffraction pattern substantially in accordance with that shown in FIG. 1, wherein the pattern is obtained from a copper radiation source (Cu—Kα 40 kV, 4 mA).

9. The method of claim 1, wherein the pharmaceutically acceptable salt of said quinolone carboxylic acid derivative is a crystalline D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate characterized by an X-ray powder diffraction pattern having peaks at about 6.35, 12.70, 19.10 and 20.50 degrees 2θ, wherein the pattern is obtained from a copper radiation source (Cu—Kα, 40 kV, 4 mA).

10. The method of claim 8, wherein the crystalline form is characterized by a melting point of about 168-171° C.

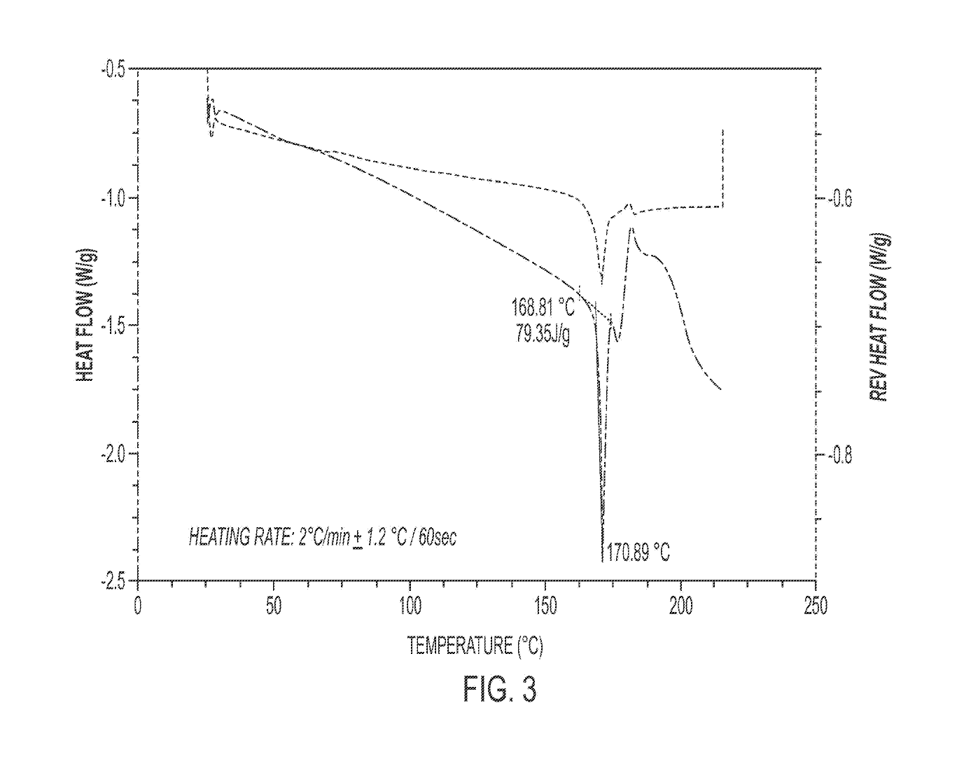

11. The method of claim 8, wherein the crystalline form is characterized by the differential scanning calorimetry thermogram shown in FIG. 3.

12. The method of claim 1, wherein said pharmaceutically acceptable salt of said quinolone carboxylic acid derivative is D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate.

13. The method of claim 1, wherein said pharmaceutically acceptable salt of said quinolone carboxylic acid derivative is crystalline D-glucitol, 1-deoxy-1-(methylamino)-, 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate, characterized, when measured at about 25° C. with Cu—Kα radiation, by the X-ray powder diffraction pattern shown in FIG. 2.

14. The method of claim 1, wherein the intravenous formulation comprises from about 100 mg to about 750 mg of said quinolone carboxylic acid derivative on an acid active basis, wherein said quinolone carboxylic acid derivative is delafloxacin or a pharmaceutically acceptable salt thereof.

15. The method of claim 14, wherein the intravenous formulation comprises about 300 mg of delafloxacin or a pharmaceutically acceptable salt thereof, on an acid active basis.

16. The method of claim 1, wherein the oral formulation comprises from about 100 mg to about 750 mg of said quinolone carboxylic acid derivative, on an acid active basis, wherein said quinolone carboxylic acid derivative is delafloxacin or a pharmaceutically acceptable salt thereof.

17. The method of claim 16, wherein the oral formulation comprises about 400 mg of delafloxacin or a pharmaceutically acceptable salt thereof, on an acid active basis.

18. The method of claim 16, wherein the oral formulation comprises about 450 mg of delafloxacin or a pharmaceutically acceptable salt thereof, on an acid active basis.

19. The method of claim 1, wherein the oral formulation is in the form of a tablet or a capsule.

20. The method of claim 19, wherein the oral formulation is in the form of a tablet and said tablet comprises:
(a) about 450 mg of said quinolone carboxylic acid derivative, wherein said quinolone carboxylic acid derivative is delafloxacin or a pharmaceutically acceptable salt thereof; and
(b) about 150 mg of an effervescent agent comprising a mixture of sodium bicarbonate, sodium dihydrogen phosphate, and citric acid.

21. The method of claim 19, wherein the oral formulation is in the form of a tablet and said tablet comprises:
(a) about 650 mg of said quinolone carboxylic acid derivative, wherein said quinolone carboxylic acid derivative is delafloxacin meglumine; and
(b) about 150 mg of an effervescent agent comprising a mixture of sodium bicarbonate, sodium dihydrogen phosphate, and citric acid.

22. The method of claim 1, wherein said intravenous administration is from about 1 to about 3 days consecutively and said oral administration is from about 1 to about 4 days consecutively.

23. The method of claim 1, wherein said intravenous administration is from about 1 to about 3 days consecutively and said oral administration is from about 2 to about 11 days consecutively.

24. The method of claim 22, wherein said intravenous formulation comprises about 300 mg of delafloxacin or a pharmaceutically acceptable salt thereof.

25. The method of claim 22, wherein said oral formulation comprises about 400 mg of delafloxacin or a pharmaceutically acceptable salt thereof.

26. The method of claim 22, wherein said oral formulation comprises about 450 mg of delafloxacin or a pharmaceutically acceptable salt thereof.

27. The method of claim 24, wherein said oral formulation comprises about 400 mg of delafloxacin or a pharmaceutically acceptable salt thereof.

28. The method of claim 24, wherein said oral formulation comprises about 450 mg of delafloxacin or a pharmaceutically acceptable salt thereof.

29. The method of claim 23, wherein said intravenous formulation comprises about 300 mg of delafloxacin or a pharmaceutically acceptable salt thereof.

30. The method of claim 23, wherein said oral formulation comprises about 400 mg of delafloxacin or a pharmaceutically acceptable salt thereof.

31. The method of claim 23, wherein said oral formulation comprises about 450 mg of delafloxacin or a pharmaceutically acceptable salt thereof.

32. The method of claim 29, wherein said oral formulation comprises about 400 mg of delafloxacin or a pharmaceutically acceptable salt thereof.

33. The method of claim 29, wherein said oral formulation comprises about 450 mg of delafloxacin or a pharmaceutically acceptable salt thereof.

34. The method of claim 1, wherein said intravenous administration is twice daily.

35. The method of claim 1, wherein the microbial infection is community acquired pneumonia.

36. The method of claim 1, wherein the microbial infection is an acute bacterial skin and skin structure infection.
